(19) United States
(12) Patent Application Publication
(10) Pub. No.: US 2007/0110726 A1
(43) Pub. Date: May 17, 2007

(54) Rust disease control by Aphanocladium album and/or Beauveria brongniartii

(75) Inventor: Ven Subbiah, Greenville, NC (US)

Correspondence Address: DANIELS DANIELS & VERDONIK, P.A., SUITE 200 GENERATION PLAZA, 1822 N.C. HIGHWAY 54 EAST, DURHAM, NC, 27713, US

(73) Assignee: PhytoMyco Research Corporation, Greenville, NC (US)

(21) Appl. No.: 11/600,433

(22) Filed: Nov. 16, 2006

Related U.S. Application Data
(60) Provisional application No. 60/737,596, filed on Nov. 17, 2005.

(51) Int. Cl. A01N 63/04; A61K 36/06

(52) U.S. Cl. 424/93.5; 424/195.16

(57) ABSTRACT

There is disclosed a method and composition for controlling rust disease in plants. Metabolites produced by Aphanocladium album mycoparasites are recovered and applied in an effective amount to plants at risk for acquiring rust disease More specifically, the application of the metabolite converts infective urediniospores that cause rust disease into non-infective teliospores. In a more specific aspect, the metabolite is reacted with another substance or under a specific reaction to result in a different compound which is also effective against rust disease.

### CROSS REFERENCE TO RELATED APPLICATIONS

This application claims priority to U.S. Provisional Application Ser. No. 60/737,596 filed Nov. 17, 2005, entitled “Rust Disease Control by Aphanocladium album, and to the filing date thereof. The disclosure of said provisional application is specifically incorporated by reference herein in its entirety.

### BACKGROUND OF THE INVENTION

1. Field of the Invention

This invention relates to methods and compositions for controlling rust disease in plants. More specifically, the plants are at least one of oat, barley, soybean, wheat, peanut, coffee, ornamental plants and pine. The method involves extracting metabolite produced by Aphanocladium album and/or Beauveria brongniartii mycoparasites and applying an effective amount to plants to prevent and or treat rust disease.

2. Background Discussion

Low molecular weight metabolites produced by the rust mycoparasite, Aphanocladium album, can convert infective urediniospores in rusts to ‘non-infective’ teliospores. Teliospore-inducing factor(s) and any associated analogs would be isolated and chemically characterized. Similarly, metabolites produced by Beauveria brongniartii can also be used. This novel mechanism of action may potentially provide an alternate approach for managing rust diseases with mycoparasite metabolites by suppressing subsequent urediniospore and rust disease development. Isolation of the teliospore-inducing factor(s) greatly facilitates basic research efforts aimed at understanding the complex life cycles of rust fungi and could potentially reveal additional mechanisms for managing rust diseases. Recent results of experiments conducted in the inventor's laboratory with A. album and its metabolites caused urediniospores of the oat rust fungus (Puccinia coronata) to convert to teliospores on excised oat leaves in the laboratory and on oat plants in greenhouse experiments. Similar observations have been shown for the wheat rust fungus, P. triticina. This early induction of non-infective teliospores will reduce disease development in the field. The ability to control the transition from the infective urediniospore to the ‘non-infective’ teliospore stage of the rust fungi provides a new and environmentally friendly method for managing rust diseases.

If teliospores are prematurely induced by mycoparasite metabolite treatment, the rust disease cycle is curtailed, thus blocking the spread of disease and lessening economic losses. Identifying the chemical structure of the teliospore-inducing compound(s) and analogs would facilitate the control of cereal rusts and would also permit the synthesis and testing of analogs. In addition, isolation of the teliospore-inducing compound(s) would greatly facilitate basic research efforts aimed at understanding the complex life cycles of the rusts. The market value for a well-defined mycoparasite metabolite with a novel mechanism of action could potentially be worth millions of dollars worldwide.

### SUMMARY OF THE INVENTION

In one aspect the invention relates to a method of controlling rust disease in plants. The method involves recovering low molecular weight metabolites produced by Aphanocladium album mycoparasites. Alternatively, such metabolites can be recovered from Beauveria brongniartii mycoparasites. An effective amount of the metabolite is applied to plants at risk of acquiring or in the early stages of rust disease to convert infective urediniospores that cause rust disease into non-infective teliospores. Yet more specifically, the plants comprise at least one of oat, barley, soybean, wheat and pine. Preferably, the metabolite comprises oosporein and can be reacted with another substance to result in diquinone prior to application to the plants. Yet still further, the diquinone may be subjected to a Thiele-Winter reaction to result in hexaacetate prior to application to the plants. In yet still another aspect, the invention relates to a composition for controlling rust disease in plants substantially as discussed above.

Thus, in summary, low molecular weight metabolites produced by the rust mycroparasite Aphanocladium album, or alternatively, Beauveria brongniartii, convert infective urediniospores to non-infective teliospores. The teliospore-inducing factor (TIF) has been isolated, identified as oosporein and demonstrated in experiments to convert infective urediniospores to non-infective teliospores of the oat, bareley, wheat, pine and soybean rust. The premature induction of non-infective teliospores by synthesized analogs of the compound provides a unique approach for the management of rust diseases. Choices of oxidant and reaction conditions allow preparation of biphenyl derivative and diquinone, both in high yield and good purity.

In an alternative approach, a Thiele-Winter reaction of diquinone is conducted to result in hexaacetate intermediates.

Cloning and characterization of genes involved in the oosporein synthesis pathway in Aphanocladium album shows that oosprein is synthesized by initial condensation of acetate units to yield orsellinic acid, which is then decarboxylated to give orcinol. Orcinol is then oxidized to produce 2,3,5-trihydroxytoluene under the function of orcinol hydroxylase in fungal cells. 2,3,5-trihydroxytoluene is further oxidized to oorsporein via acetylpyruvate under the function of 2,3 5-trihydroxytoluene 1,2-oxygenase in fungal cells. Orcinol and 2,3,5-trihydroxytoluene are important intermediates and orcinol hydroxylase and 2,3,5-trihydroxytoluene 1,2-oxygenase are important enzymes in the oosporein biosynthesis pathway.

In accordance with the invention, the total amount of inoculum produced per uredinium depends on the number of urediniospores formed, how rapidly they form, and the duration of active sporulation. Epidemics may be suppressed by limiting the total inoculum generated by an individual uredinium. Urediniospore formation is terminated by the death of the surrounding host tissue or by conversion to the teliospore phase. The premature replacement of urediospores by teliospores breaks the disease cycle. Thus, premature induction of ‘non-infective’ teliospores by applying either the metabolites or the pure compound from A. album showed novel opportunity for managing rust diseases of cereals. The teliospores can only infect the alternate (non-economic) hosts, which are non-existent in the southern U.S. where rust diseases cause the most crop damage. Thus, a rapid and early conversion to the telia stage renders the pathogen unable to re-infect the economically important cereal crops.

Low molecular weight metabolites and the pure compound isolated from the rust mycoparasite, Aphanocladium album (Aa 100), converted infective urediniospores in rusts to ‘non-infective’ teliospores with in three days of application. Using assay-guided fractionation, we successfully isolated the pure compound and established the telia-inducing activity. Subsequently using different spectroscopy (LC-MS, NMR) we have identified the chemical structure of the active compound. This novel mechanism of action may potentially provide an alternate approach for managing rust diseases. Using the compound or its analogs we could suppress subsequent urediniospore formation and rust disease development. We hypothesize that this early induction of non-infective teliospores would reduce disease development in the field. The ability to control the transition from the infective urediniospore to the ‘non-infective’ teliospore stage of the rust fungi would provide a new and environmentally friendly method for managing rust diseases. By inducing teliospores prematurely by mycoparasite metabolite or pure compound treatment, the rust disease cycle would be curtailed, thus blocking the spread of disease and lessening economic losses. Preparation of synthetic analogs based on the structure of the pure compound would greatly facilitate the control of cereal rusts more effectively in the field conditions.

Separately, Beauveria brongniartii can be used to extract metabolites useful in accordance with the invention.

In one aspect, there is disclosed: i) studying of fermentation of A. album to produce kilo quantity of the compound; ii) initiating synthetic work around the structure to prepare analogs to enhance the telia-inducing properties; iii) conducting field studies using the compound or its synthetic analogs; and iv) formulating the active compound for evaluation under field conditions.

Soybean rust has been found in every other major producing nation and as of the Fall of 2004, soybean rust had been found in the continental United States. The invention involves applying the compounds in particular on soybean rust, as well as other plants.

### BRIEF DESCRIPTION OF THE SEVERAL VIEWS OF THE DRAWING

FIG. 1 is a color photograph showing symptoms of infection on wheat leaves 4 days after inoculation with urediniospores of Puccinia coronata;

FIG. 2 is a color photograph showing Uridea and urediniospores of Puccinia coronata on upper leaf surfaces of oat seedlings;

FIG. 3 is color photograph showing Uridea and urediniospores of Puccinia coronata on lower leaf surfaces of oat seedlings treated with Aphanocladium album (Aa100) crude extract;

FIGS. 4 and 5 are color photographs showing formation of telia on oat seedlings treated with Aphanocladium album (Aa100) crude extract;

FIG. 6 is a color photograph showing formulation of telia on oat seedlings treated with Aphanocladium album (Aa100) service-purified fraction (Aa100-II-3);

FIG. 7 is a color photograph showing formation of telia on oat seedlings treated with pure compound (Oosporein) isolated from Aphanocladium album (Aa100);

FIG. 8 is a color photograph showing teliospores and urediniospores of Puccinea coronata;

FIG. 9 is a chromatogram for a sample Aa100-II-3 analyzed in positive ion electrospray LC/MS/MS;

FIG. 10 is a graph showing LC/MS and LC/MS/MS positive ion electrospray nms spectra for peaks at retention time of 1.4 minutes and 2.1 minutes;

FIG. 11 is a graph showing LC/MS and LC/MS/MS positive ion electrospray nms spectra for peaks at retention time of 5.0 minutes and 5.5 minutes;

FIG. 12 is a graph showing LC/MS and LC/MS/MS positive ion electrospray nms spectra for peaks at retention time of 5.6 minutes and 5.8 minutes;

FIG. 13 is a graph showing LC/MS and LC/MS/MS positive ion electrospray nms spectra for peaks at retention time of 6.4 minutes and 6.5 minutes;

FIG. 14 is a graph showing LC/MS and LC/MS/MS positive ion electrospray nms spectra for peaks at retention time of 8.0 minutes and 8.2 minutes;

FIG. 15 is a UV at 254 nm and total ion chromatogram for a sample Aa100-II-3 analyzed in negative ion slectropray LC/MS/MS;

FIG. 16 is a graph of LC/MS and LC/MS/MS negative ion electrospray nms spectra for peaks at a relation time of 0.5 minutes and 0.6 minutes;

FIG. 17 is a graph of LC/MS and LC/MS/MS negative ion electrospray nms-spectra for peaks at a relation time of 0.7 minutes and 0.8 minutes; and

FIG. 18 is a graph of LC/MS and LC/MS/MS negative ion electrospray nms spectra for peaks at a relation time of 0.8 minutes and 0.9 minutes.

### DETAILED DESCRIPTION OF THE INVENTION

Cloning and characterization of genes involved in the oosporein synthesis pathway Oosporein is a C2 symmetrical red 2,5-dihydroxybenzoquinone derivative biosynthesized by a broad variety of soil borne fungi such as Aphanocladium album and Beauveria brongniartii. It is the major secreted secondary metabolite of these enthomopathogenic fungi. Oosporein is used as a selective and highly virulent biocontrol agent (BCA) against Melolontha melolontha L. and other rust diseases. Oosporein has been known for almost six decade. However, how oosporein is biologically synthesized in fungal cells is not completely documented. Genes encode enzymes that are involved in the oosporein biosynthesis pathway are not cloned and identified.

It is believed that oosporein is synthesized by initial condensation of acetate units to yield orsellinic acid, which is then decarboxylated to give orcinol. Orcinol is then oxidized to produce 2,3,5-trihydroxytoluene under the function of orcinol hydroxylase in fungal cells. 2,3,5-trihydroxytoluene is further oxidized to oosporein via an unknown enzyme or mechanism or form acetylpyruvate under the function of 2,3,5-trihydroxytoluene 1,2-oxygenase in fungal cells. Orcinol and 2,3,5-trihydroxytoluene are very important intermediates and orcinal hydroxylase and 2,3,5-trihydroxytoluene 1,2-oxygenase are very important enzymes in the oosporein biosynthesis pathway.

Based on the functional characterization of orcinal hydroxylase and 2,3,5-trihydroxytoluene 1,2-oxygenase in Pseudomonas putida, increasing the content of orcinal hydroxylase will results in increased accumulation of 2,3,5-trihydroxytoluene in cell cultures, and decreasing 2,3,5-trihydroxytoluene 1,2-oxygenase will inhibit the decomposing of 2,3,5-trihydroxytoluene into acetylpyruvate and then indirectly increase oosporein biosynthesis. Orcinal hydroxylase and 2,3,5-trihydroxytoluene 1,2-oxygenase have been purified and identified from Pseudomonas putida (Ohta et al., 1975). Their physical and catalytic properties have been reported (Ohta et al. 1975). However, genes controlled the synthesis of orcinal hydroxylase and 2,3,5-trihydroxytoluene 1,2-oxygenase have not been isolated and identified from any species. In accordance with the invention, we propose to clone orcinal hydroxylase and 2,3,5-trihydroxytoluene 1,2-oxygenase genes are cloned from the Aphanocladium album.

Orcinol and 2,3,5-trihydroxytoluene are important intermediates in the oosporein biosynthesis pathway. Metabolism of orcinol has been identified in two strains of Pseudomonas putida that are capable of growth with orcinol (3,5-dihydroxytoluene) as the sole source of carbon. Experiments with cell suspensions and cell extracts indicated that orcinol is metabolized by hydroxylation of the benzene ring followed successively by ring cleavage and hydrolyses to give 2 mol of acetate and 1 mol of pyruvate per mol of orcinol as shown: orcinol→2,3,5-trihydroxytoluene→2,4,6-trioxoheptanoate→acetate+acetylpyruvate→acetate+pyruvate. Evidence for this pathway was based on: (i) high respiratory activities of orcinol-grown cells towards 2,3,5-trihydroxytoluene; (ii) transient accumulation of a quinone, probably 2-hydroxy-6-methyl-1,4-benzoquinone, during growth with orcinol; (iii) formation of pyruvate and acetate from orcinol, 2,3,5-trihydroxytoluene, and acetylpyruvate catalyzed by extracts of orcinol, but not by succinate-grown cells; (iv) characterization of the product of oxidation of 3-methylcatechol (an analogue of 2,3,5-trihydroxytoluene) showing that oxygenative cleavage occurs between carbons bearing methyl and hydroxyl substituents; (v) transient appearance of a compound having spectral properties similar to those of acetylpyruvate during 2,3,5-trihydroxytoluene oxidation by extracts of orcinol-grown cell. Orcinol hydroxylase exhibits catalytic activity when resorcinol or m-cresol is substituted for orcinol; hydroxyquinol and 3-methylcatechol are substrates for the ring cleavage enzyme 2,3,5-trihydroxytoluene-1,2-oxygenase.

The following shows the oosporein biosynthesis pathway of the invention.

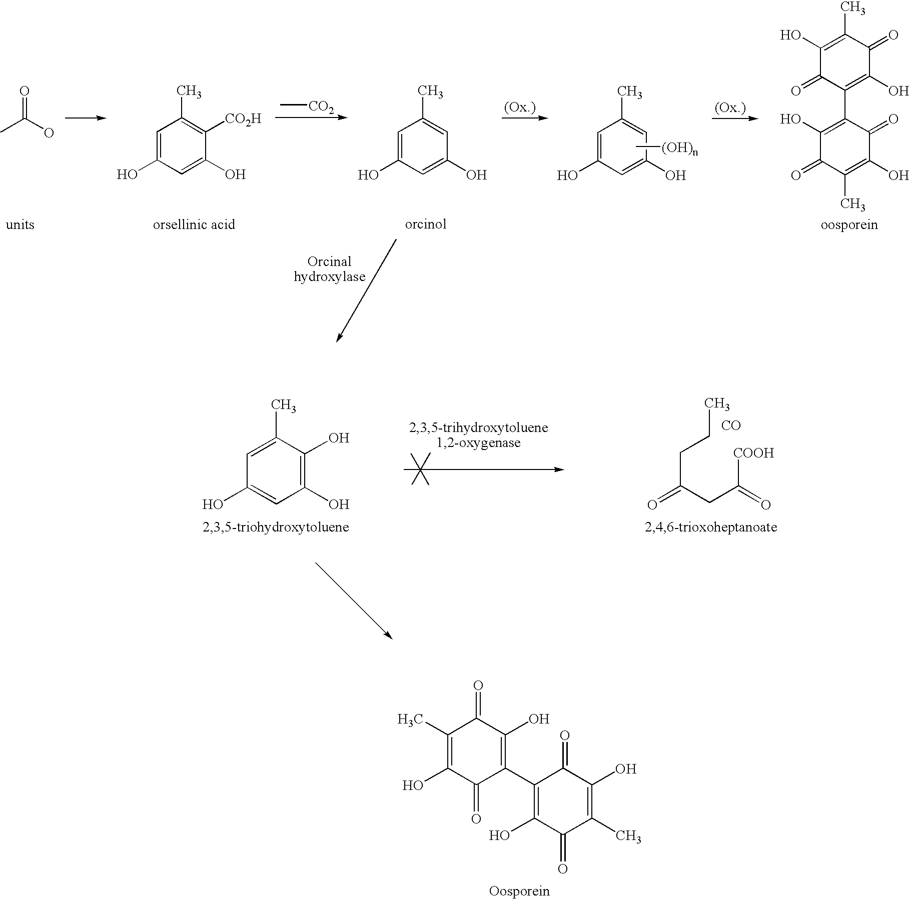

The invention further involves the use of mycoparasite metabolites for managing rust diseases, particularly focusing on the secondary metabolites produced by the mycoparasite, Aphanocladium album and Beauveria brongniartii to control cereal rusts.

The following are specific examples of the invention.

1. Evaluation of Secondary Metabolites from Aphanocladium album for Early Induction of Telia of the Oat Rust Fungus Puccinia coronata

Several initial experiments were conducted to optimize the conditions in laboratory in vitro, and greenhouse in vivo experiments. Several solubility and treatment methods were used and finally optimal conditions were determined for seedling stage, application time, valuation cycle and other climatic conditions. For this report only the final results have selected. Following are some of the details on plant varieties, rust isolates and the mycoparasite (Aphanocladium album) isolates:

i) Oat variety ‘Brooks’

ii) Two rust isolates were used, one from Minnesota the other from North Carolina

iii) Three A. album isolates were tested (Aa 100. Aa 5998 and Aa 6433), however, for Aa 100 was the major culture used for bulk fermentation, fractionation and chemical isolation

iv) Both crude extracts and fractions were tested at 10 mg/ml and the pure compound at 100 μg/ml

1a. Greenhouse Experiments with Crude Extracts of A. album Isolates (Aa 100 and Aa 5988)

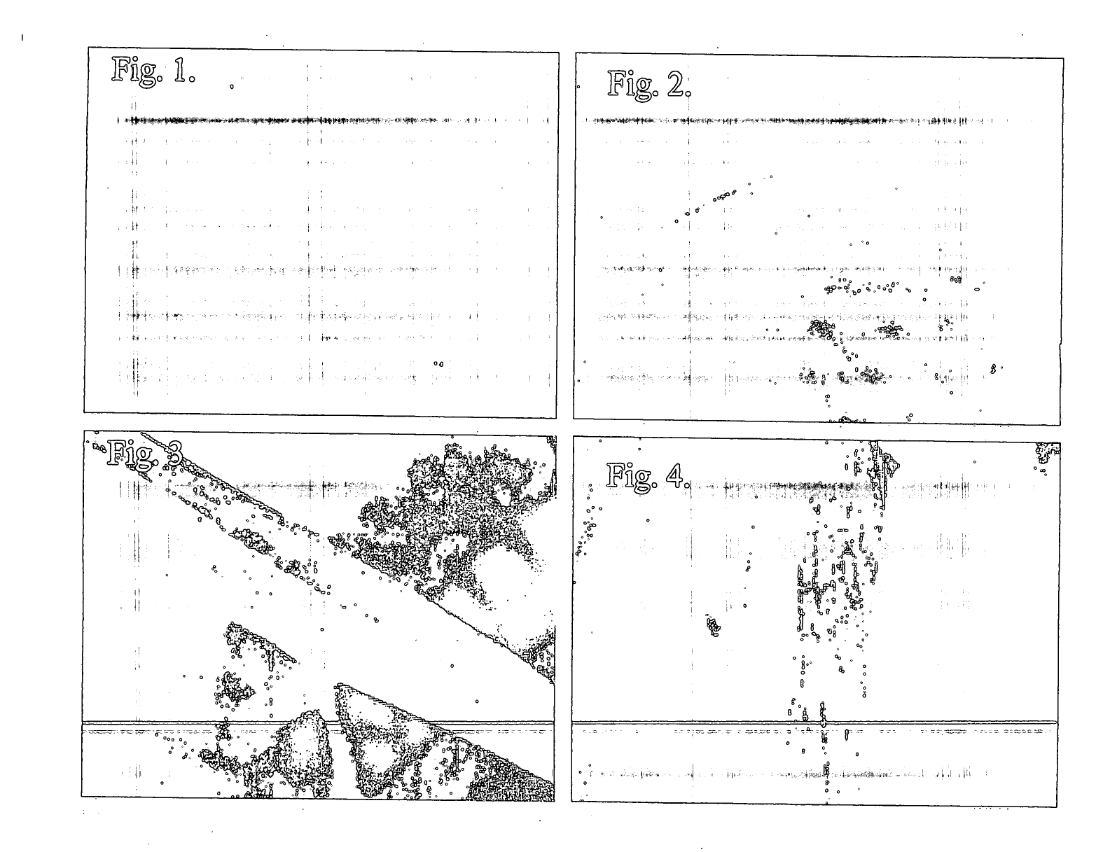

The culture extract from two isolates of A. album (Aa 100 and Aa 5998) were dissolved in 500 pt of DMSO and 9.5 ml of sterile distilled water (SDW) with 20 μl of Tween 80. The concentration of Aa 100 and Aa 5988. Controls consisted of 500 μl of DMSO in 9.5 ml of SDW with 20 pt of Tween 80. and 10 ml of SDW with 20 pt of Tween 80. Ten ml of each treatment were atomized on 2-3 week-old, oat seedlings previously inoculated with strain Minnesota (MN) of Puccinia coronata. Treatments were applied four days after inoculation when flecking symptoms of infection were present, but before uredia were observed (FIG. 1). There were five replicates of each treatment arranged in a randomized complete block design.

Results—Uredia and urediospores were observed with in six days (FIG. 2) and telia were observed on the 8^{th} day (FIG. 3). Oat seedlings sprayed with Aa 100 had a higher percentage of plants with telia than the other treatments (Table 1). However, these differences were not significantly different (P=0.05). Ten days after application of treatment Aa 100, the intensity of telial on oat leaves was significantly higher than Aa 5998 and the water control.

TABLE 1
Percentage of oat (cv. Brooks) seedlings with telia
and intensity of telial formation on seedlings inoculated
with strain MN of Puccinia coronata

| | Percentage of | Telia intensity | |
|---|---|---|---|
| Treatment | plants with telia¹ | Rating^{2/} | |
| Aa 100 extract | 60 | 1.3 | a |
| Aa 5988 | 30 | 0.5 | bc |
| Control | 25 | 0.2 | c |
| FLSD(P = 0.05) | ns | 0.7 | |
| CV (%) | 66 | 76 | |

¹Values represent the mean of five replicates of four seedlings per pot.
^{2/}Values represent the mean of five replicates of four seedlings per pot and were determined based on the following scale; 0 = healthy; 0.5 = <2% of leaf area with telia; 1 = 3-5% of leaf area with telia;. 1.5 = 6-8% of leaf area with telia; 2.0 = 9-11% of leaf area with telia; 2.5 = 12-14% of leaf area with telia; 3.0 = >15% of leaf area with telia..

1.b. Greenhouse Experiments with Active Fraction of A. album Isolates (Aa 100-II-3 and Aa 5988)

The culture extract from two isolates of A. album (Aa 100 and Aa 5998) were dissolved in 500 μl of DMSO and 9.5 ml of sterile distilled water (SDW) with 20 μl of Tween 80. Controls consisted of 500 pt of DMSO in 9.5 ml of SDW with 20 μl of Tween 80. and 10 ml of SDW with 20 μl of Tween 80. Ten ml of each treatment were atomized on 2-3 week-old, oat seedlings previously inoculated with strain NC of Puccinia coronata. Treatments were applied four days after inoculation when flecking symptoms of infection were present, but before uredia were observed (FIG. 1). There were five replicates of each treatment arranged in a randomized complete block design.

Results—Uredia and urediospores were observed on 7^{th} day and telia were observed on the 9^{th} day (FIGS. 4 and 5). Oat seedlings sprayed with Aa 100 fractions had a significantly higher percentage of plants with telia and with a greater intensity of telia than the other treatments (Table 2).

TABLE 2
Percentage of oat (cv. Brooks) seedlings with telia
and intensity of telial formation on seedlings inoculated
with strain NC of Puccinia coronata

| | | |
|---|---|---|
| | Percentage of | Telia intensity |
| Treatment | plants with telia^{1/} | Rating^{2/} |

| | | | | |
|---|---|---|---|---|
| Aa 100 Fraction | 85 | a | 1.9 | a |
| Aa 5988 fraction | 25 | a | 0.7 | b |
| Control | 25 | a | 0.3 | b |
| FLSD(P = 0.05) | 40 | | 1.1 | |
| CV (%) | 77 | | 96 | |

^{1/}Values represent the mean of five replicates of four seedlings per pot.
^{2/}Values represent the mean of five replicates of four seedlings per pot and were determined based on the following scale; 0 = healthy; 0.5 = <2% of leaf area with telia; 1 = 3-5% of leaf area with telia;. 1.5 = 6-8% of leaf area with telia; 2.0 = 9-11% of leaf area with telia; 2.5 = 12-14% of leaf area with telia; 3.0 = >15% of leaf area with telia..

1.c. Greenhouse Experiments with the Pure Compound, Oosporein Isolated from of A. album (Aa 100 Isolate):

The pure compound (Oosporein) isolated from A. album (Aa 100) dissolved in 500 μl of DMSO and 9.5 ml of sterile distilled water (SDW) with 20 μl of Tween 80. The controls consisted of 500 μl of DMSO in 9.5 ml of SDW with 20 μl of Tween 80. and 10 ml of SDW with 20 μl of Tween 80. Ten ml of each treatment were atomized on 2-3 week-old, oat seedlings previously inoculated with strain MN of Puccinia coronata. Treatments were applied four days after inoculation when flecking symptoms of infection were present, but before uredia were observed (FIG. 1). There were five replicates of each treatment arranged in a randomized complete block design.

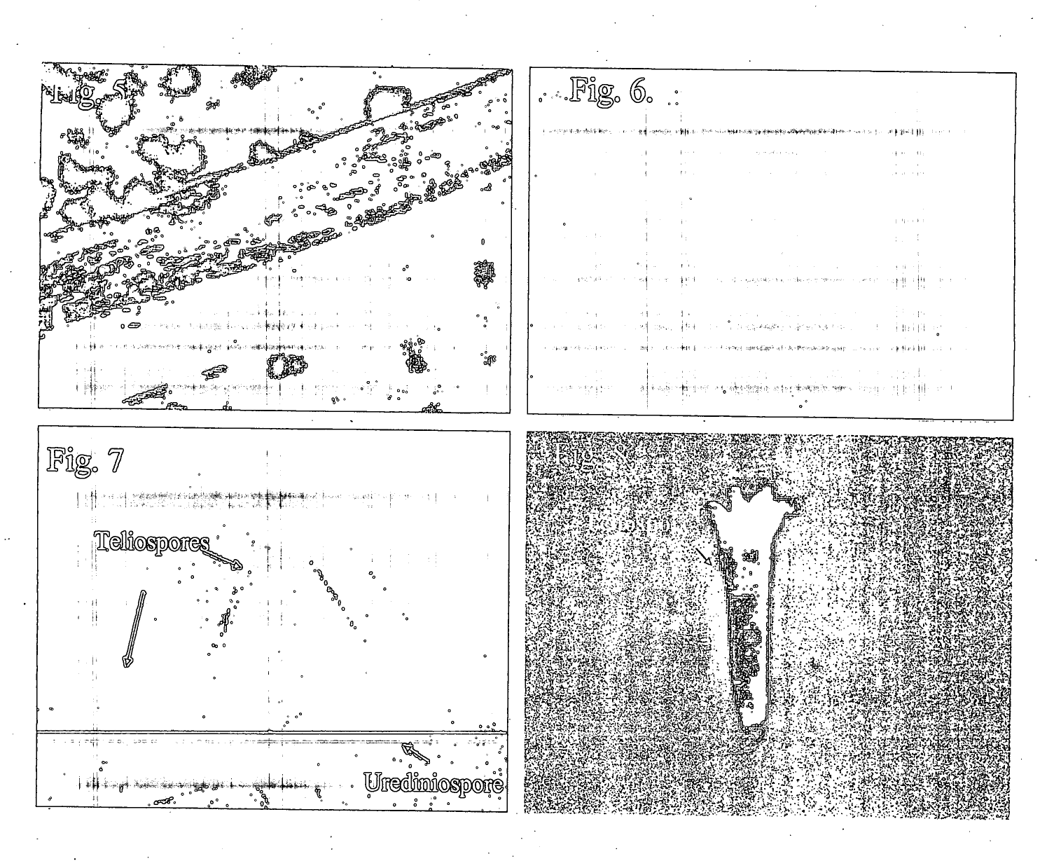

Results—Uredia and urediospores were observed on the 7^{th} day and telia were observed on the 9^{th} day (FIG. 6). Oat seedlings sprayed with Ooosporein had a higher percentage of plants with telia and a greater intensity of telia than the other treatments (Table 3).

TABLE 3
Percentage of oat (cv. Brooks) seedlings with telia
and intensity of telial formation on seedlings inoculated
with strain NC of Puccinia coronata

| | Percentage of | Telia intensity |
|---|---|---|
| Treatment | plants with telia¹ | Rating^{2/} |
| Oosporein | 83 | 8.2 |
| Control | 37 | 0.5 |
| FLSD(P = 0.05) | ns | ns |
| CV (%) | 68 | 64 |

¹Values represent the mean of five replicates of four seedlings per pot.
^{2/}Values represent the mean of five replicates of four seedlings per pot and were determined based on the following scale; 0 = healthy; 0.5 = <2% of leaf area with telia; 1 = 3-5% of leaf area with telia;. 1.5 = 6-8% of leaf area with telia; 2.0 = 9-11% of leaf area with telia; 2.5 = 12-14% of leaf area with telia; 3.0 = >15% of leaf area with telia..

Microscopic Observation of the Transverse Section of the Infected Leaf with Teliospores:

Transverse sections made from the infected leaves (black telio spore covered) showed enormous number of characteristic two celled, crown shaped teliospores (FIGS. 7 and 8).

2. Scaling-Up A. album Fermentation, Isolating Preparative Quantities of Crude Active Fractions, and Purifying Active Components Chromatographically:

Applying experience in microbial strain improvement and nutritional requirements, several media recipes that included both liquid and solid-state fermentation were tried. We also monitored the activity of the culture filtrate periodically to optimize the fermentation time and other conditions. The invention involved scaled-up ten liter fermentation capacity and optimized all conditions required to obtain large quantity of crude secondary metabolites to facilitate the isolation and identification of the active compound(s). Several media and absorptive gels were tried before the scale-up level. The bulk fermented product was extracted and used for the isolation of the active compound using different chromatography and spectroscopy techniques.

3. Isolating and Identifying Teliospore-Inducing Factor(S) from A. album:

In one aspect the invention involves characterizing the metabolite(s) from A. album and determining the structure of the active compound (chemical structure). The metabolite has been isolated and two more isolates of A. album added to the study. Comparative studies with the culture filtrate as well as the crude extract showed that the original culture (Aa 100) is more virulent than the other two new isolates (Aa 5998 and Aa 6433). For subsequent work, isolate Aa 100 was used.

Several fermentation combinations were developed to enhance the production of secondary metabolites. The activity was assessed by in vitro detached leaf assays. A combination of V8-based potato dextrose medium has supported the maximum growth and secretion of the secondary metabolites.

Several fractions (enriched compounds) were isolated and the invention involved performing assay-guided purification as well as identification by LC-MS (Liquid chromatography-Mass spectroscopy) and other spectroscopy (NMR and MS). Use was made of the dereplication (a powerful tool to identify the known compound early on) based on HPLC-diode array detector and 3D Chem Station and external database (Chapman and Hall natural product database) to narrow down the identification process. These two systems were very useful in the detection of previously characterized natural product compounds. The Diode-array detector with Chem Station is useful in measuring UV absorption spectra in one injection, checking for peak purity and confirming the identity of a peak through comparative analysis of the UV spectra. This tremendously helped to reduce cumbersome, labor intensive and costly investigations into previously characterized natural product compounds.

Based on the assay-guided fractionation (telia inducing property) one single compound has been identified. Using different chromatography (flash column chromatography, TLC and HPLC), the compound was purified to homogeneity. Its purity was tested on HPLC and was subjected to nondestructive spectrochemical analysis using proton and 13C nuclear magnetic resonance techniques. Additional experiments such as infrared and UV-VIS spectra. Low resolution chemical ionization and electron impact spectra was obtained using a Hewlett-Packard 5985-B GC-MS. High resolution mass spectra and elemental composition was determined on an A. E. I. MS-902 mass spectrometer.

3a. LC/MS/MS Analysis of Sample Aa 100-II-3

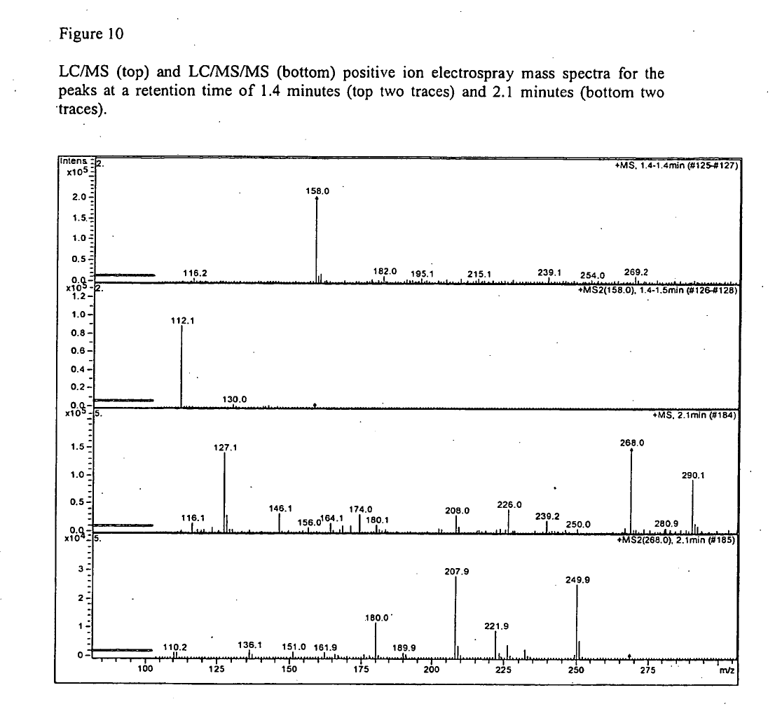

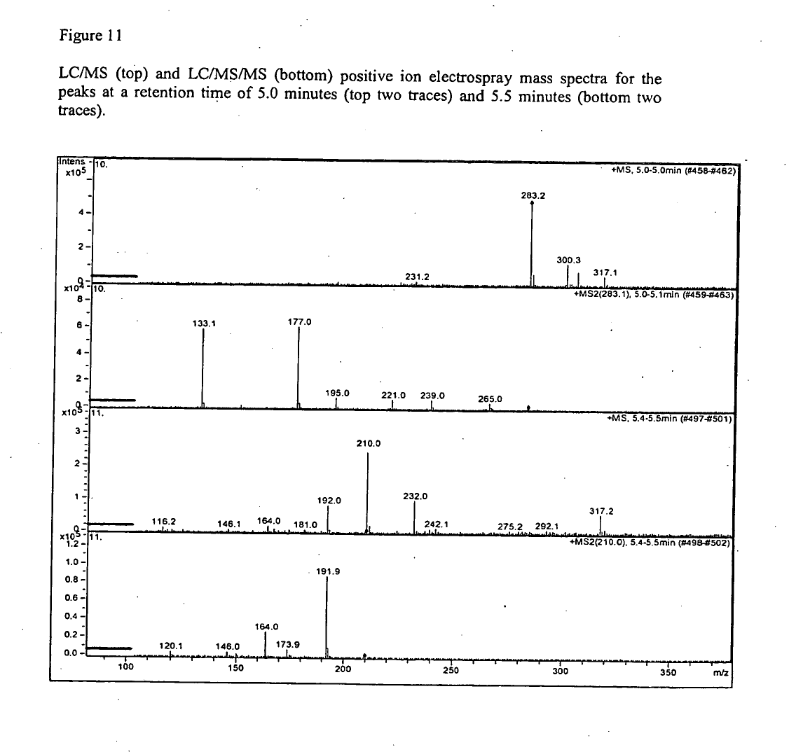

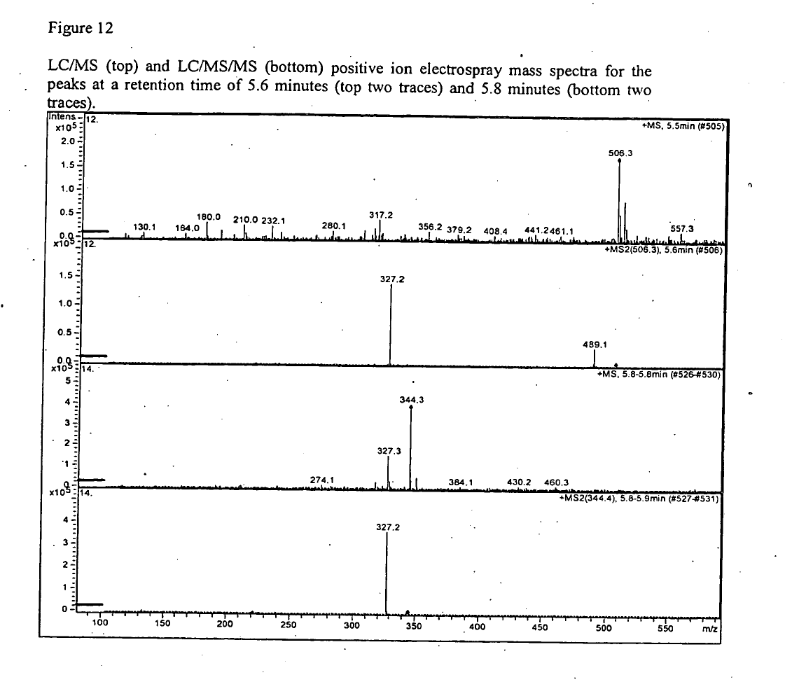

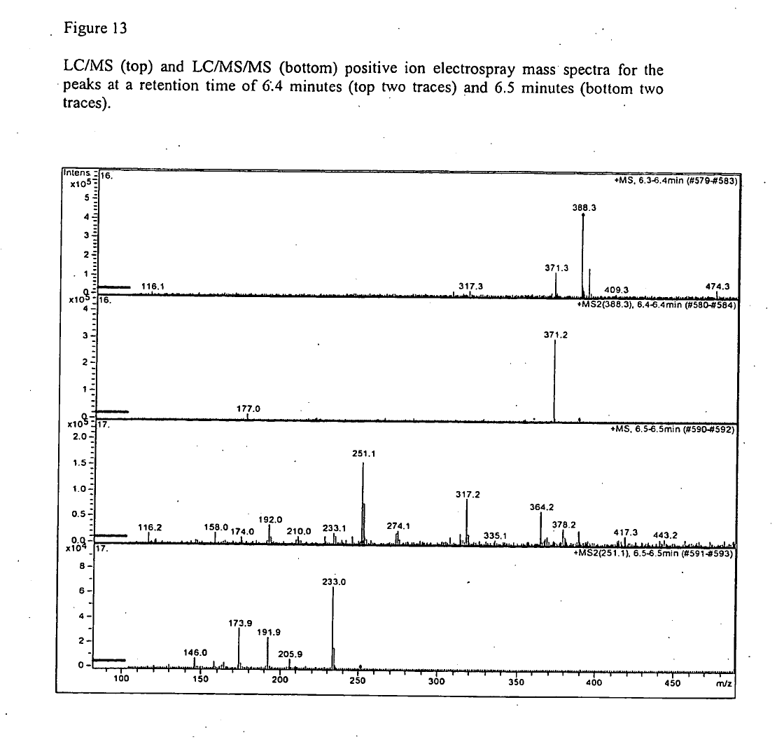

The LC/MS/MS data was acquired for the A. album active fraction (Aa 100-II-3) using both electrospray positive and negative ion detection. The LC/MS conditions used for the analysis are given in Table 4. FIG. 1 shows the UV and total ion current chromatogram for the electrospray positive ion LC/MS/MS analysis. FIGS. 8-12 shown hereafter present the LC/MS mass spectra for the ten most intense peaks in the chromatogram. FIG. 13 shows the UV and total ion current chromatogram for the electrospray negative ion LC/MS/MS analysis. FIGS. 8-10 present the negative ion LC/MS mass spectra for the most intense peaks in the chromatogram.

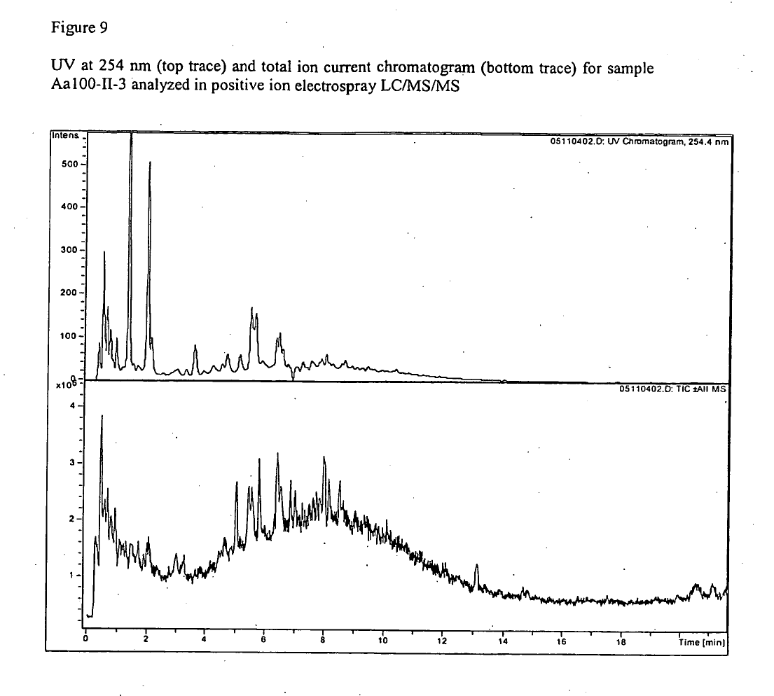

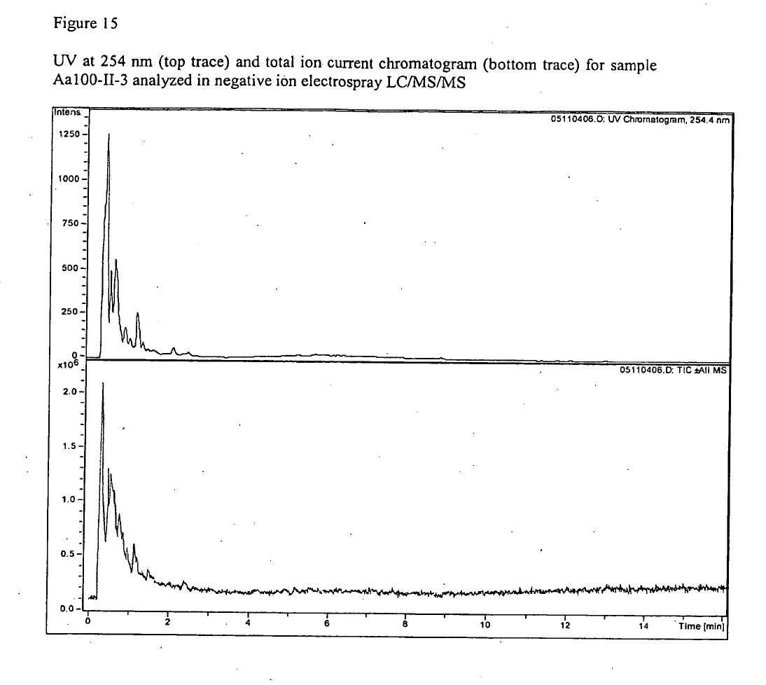

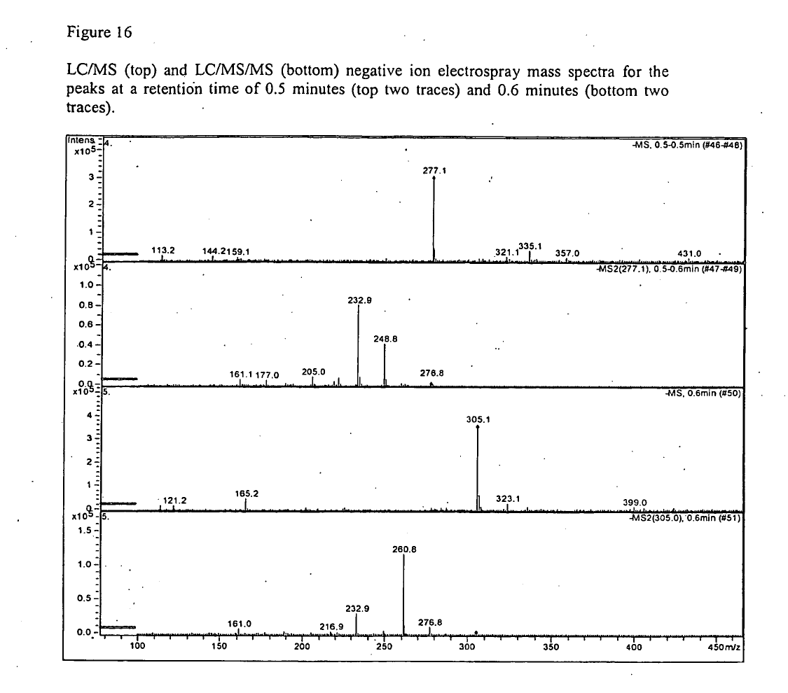

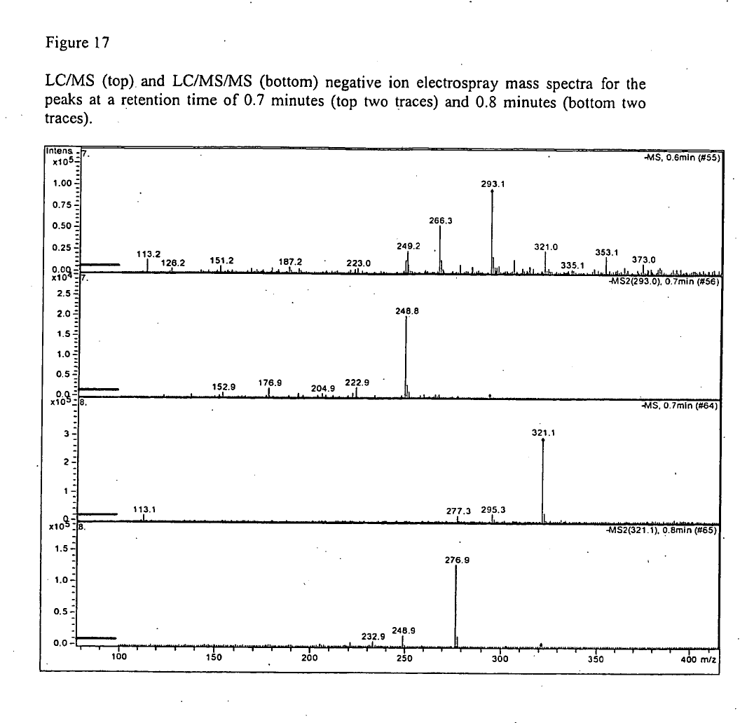

Table 5 summarizes the data presented in FIGS. 9-18. The components in the sample range in molecular weight between 157-505. Based on the MS/MS data, any components contained a carboxylic acid, based on losses of CO₂, CO and H₂O in the MS/MS mass spectra. All compounds detected in negative ion were from carboxylic acids. Also, there were several amine containing components detected.

TABLE 4
Experimental LC/MS Conditions

LC conditions:
Column: Agilent Zorbax SBC₁₈ 2.1 × 50 mm × 3.5 um particles
Gradient: 0% B to 100% B in 30 minutes
A = 5% acetonitrile/95% water (0.025% TFA)
B = 95% acetonitrile/5% water (0.025% TFA)
For negative ion detection the TFA was switched for
25 nM ammonium acetate
Flowrate: 0.3 mL/min
Injection volume: 4 uL
Analysis time: 30 min with a 8 min equilibration time
UV detector: 254 nm
MS Conditions:
Mode of operation: Positive ion and negative ion electrospray on
Agilent 1100 MSD Trap system
Scan range: 100-1000
Mode: Auto MS/MS of most intense ion above 100,000 counts
MS/MS Conditions: Helium collision gas, cutoff 35% of parent,
1.2 V CID energy for 30 ms
Capillary exit voltage: 70 V
Drying gas: 9.5 L/min at 350 C.
Nebulization pressure: 45 psi

TABLE 5
Summary of components found by LC/MS in sample Aa100-II-3

| | | |
|---|---|---|
| Ret time (min) | Mol wt | MS/MS losses |

Positive ion detection

| | | |
|---|---|---|
| 1.4 | 157 | loss of 46 (water and CO) |
| 2.1 | 267 | losses of water, acetic acid and CO |
| 5.0 | 282 | losses of 106 and CO2 |
| 5.4 | 209 | losses of water, and CO |
| 5.5 | 505 | losses of NH3 and 179 |
| 5.8 | 343 | losses of NH3 |
| 6.3 | 387 | losses of NH3 |
| 6.5 | 250 | losses of water |
| 8.0 | 392 | ions at 357, 333, 315, 287 and 221 |
| 8.2 | 375 | losses of water, acetate and CO |

negative ion detection

| | | |
|---|---|---|
| 0.5 | 278 | losses of CO2 and CO |
| 0.6 | 306 | losses of CO2 and CO |
| 0.7 | 294 | losses of CO2 |
| 0.8 | 322 | losses of CO2 and CO |
| 0.8 | 296 | losses of CO2 and CO |
| 0.9 | 280 | losses of CO2 and H2O |
| 1.5 | 356 | losses of CO2, H2O and CO |

3b. LC-MS Confirmation of Oosporein and Quantification:

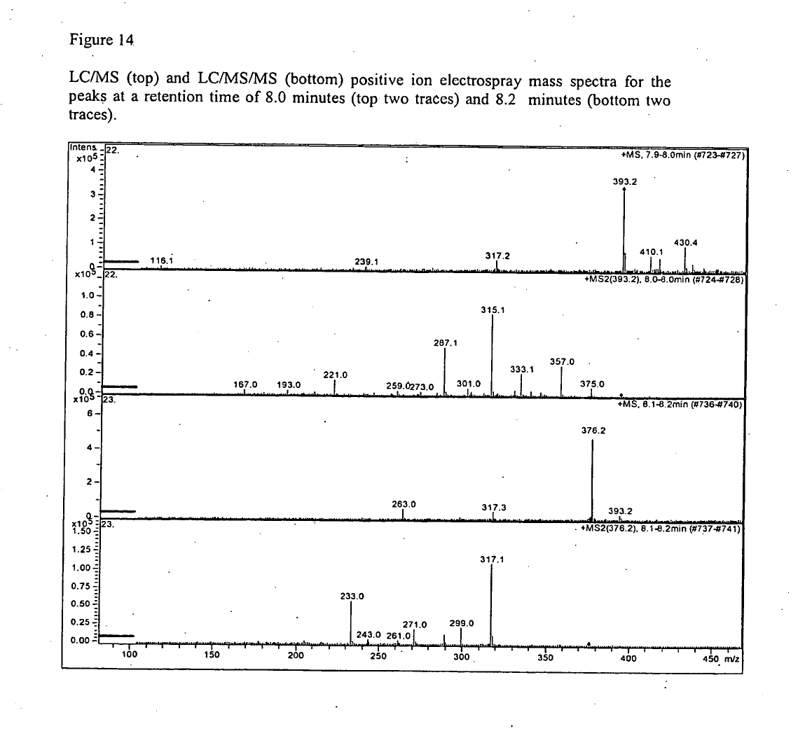

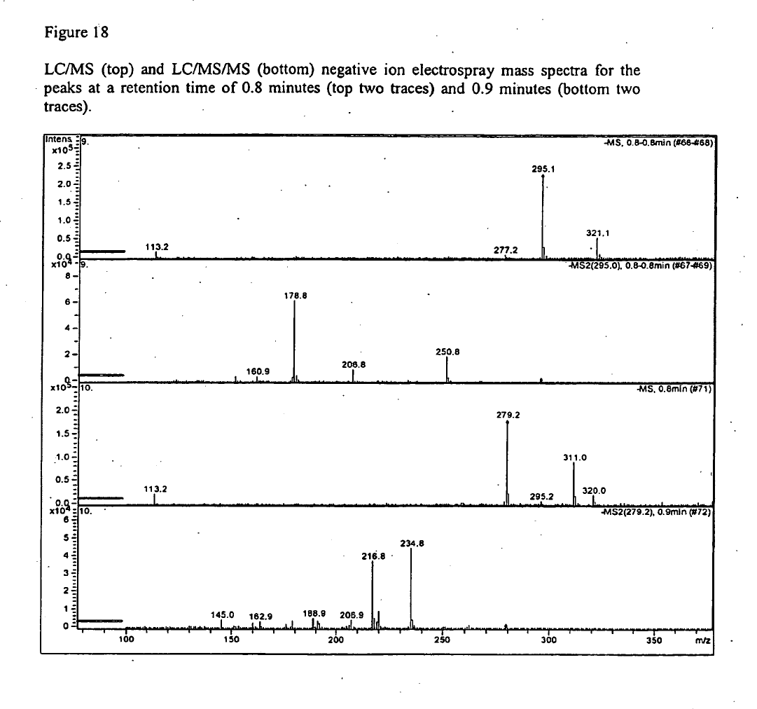

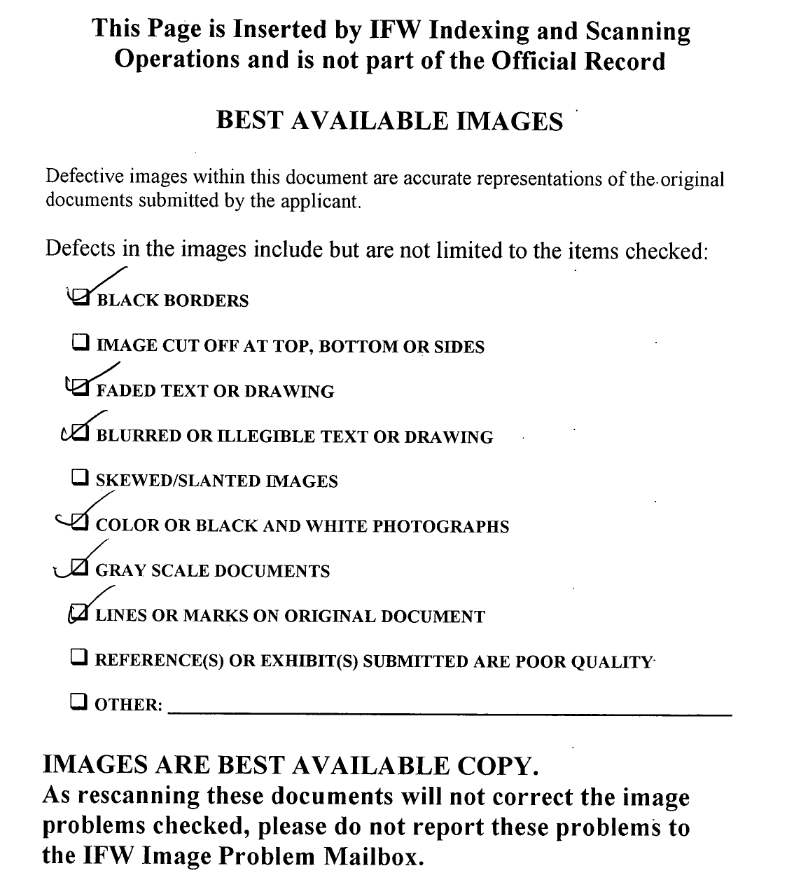

The LC/MS/MS data acquired from the standard of Oosporein using the same conditions as reported before. FIG. 18 shows the extracted ion current chromatogram and MS and MS/MS spectrum for the standard of Oosporein. The spectrum matches the one reported in the bottom of FIG. 14, confirming the presence of Oosporein in sample Aa100-II-3.

Comparing the peak area for a 1.7 mg/ml solution of Oosporein (19,960,601 counts) to a solution of Aa100-II-3 (988,100 counts) for the same injection volume, the estimated amount of Oosporein is 0.084 mg/ml. The Aa100-II-3 solution was made up as a 10 mg/ml solution giving a weight percentage of 0.84%.

3c. Nuclear Magnetic Resonance (NMR) to Confirm the Structure of Ooosporein:

Identification was also confirmed based on nuclear magnetic resonance (NMR). The NMR spectra were measured on a GE500 Omega spectrometer in D Acetone. The confirmation was based on comparison with published papers (FIG. 19).

Cytotoxicity Assay for Oosporein:

di-Methylthiozol Tetrazolium (MTT assay): Proliferation assays are used in cell biology for the study of growth factors, cytokines and cytotoxic agents. There are several ways to measure the number of cells in a proliferation assay. MTT assay is a rapid and convenient method of determining viable cells in proliferation, cell attachment, chemotaxis and apoptosis assays.

di-Methylthiozol Tetrazolium Salt (MTS assay): MTS assay is a rapid and convenient method of determining viable cells in proliferation or cytotoxic assays. The use of this analog of MTT, provides a stable reaction over time. MTS assay is based on the cellular conversion of a tetrazolium salt into a formozan product that is soluble in tissue culture media and can easily be detected using a 96-well plate reader. This conversion is presumably accomplished by NADPH or NADH produced by dehydrogenases enzymes present in metabolically active cells. MTS is preferred over MTT because the MTS formazan product is soluble in tissue culture medium. In addition the MTS assay require fewer steps than MTT assay. Consequently, we have used MTS for our testing.
MTS assay-Method: K-562 (typical cancer cell line used for cytotoxicity assays) cells were seeded in 96-well plates at a concentration of 2.5×10⁴ cells/ml. Oosporein and Actinomycin D (a known cytotoxic compound) were prepared in 10% DMSO and added to cells the next day (final DMSO concentration was 0.5%). Oosporein was assessed at a final concentration on the cells of 100, 50, 25, 12.5, 6.25 and 3.125 μg/ml (2-fold dilutions) in quadruplicate. A known toxic agent, Actinomycin D, was added at 100, 20, 4, 0.8 and 0.16 ng/ml (5-fold dilutions). After 3 days exposure to test compound or controls, MTS reagent was prepared and added to the cells. After 3 hours, the accumulation of formazan product was determined by measuring the absorbance at 490 nm. The background absorbance of the cells detected at 630 nm was then subtracted from the initial 490 nm reading. Percent toxicity was determined using the following formula:

$$\% Toxicity = ( sample - blank ) / ( vehicle - blank ) \times 100$$

$$Confidence levels calculated at p < .05 .$$

Results: The MTS assay method used is highly sensitive for toxic effects because relatively few cells are seeded per well, and the cells are exposed for three whole days to test compounds. It is important to also point out that while Oosporein and Actinomycin D are both pure compounds, they were compared at different concentration (μg vs ng respectively). Oosporein showed very mild toxicity on K-562 cells at the highest concentrations tested, 100 and 50 μg/ml. Interestingly, there was no increased toxic effect at 100 μg/ml vs. 50 μg/ml. Actinomycin D showed similar levels of toxicity on cells when tested at 20 ng/ml, and almost 100% toxicity at a concentration of 100 ng/ml (500× less concentrated than Oosporein). However, Oosporein exhibited no toxicity whatsoever when given to the cells at concentrations of 25 μg/ml or less. These results indicate that Oosporein exhibited mild toxic effects on K-562 cells at very high concentrations (as would most pure compounds tested at this concentration level), Oosporein had no toxicity on cells when used at concentrations tested for inducing telio spores on oat plants. [image]
Compound Structure Legends to Figures Showing the Telio-Inducing Activity of A. Album Metabolites on Wheat Leaves in Greenhouse Conditions
FIG. 1. Symptoms of infection 4 days after inoculation with urediospores of Puccinia coronata. Treatments were applied when yellow flecking occurred on leaves
FIG. 2. Uredia and urediospores of Puccinia coronata on upper leaf surface of oat seedlings (cv. ‘Brooks’)
FIGS. 3-4. Uredia and urediospores of Puccinia coronata on lower leaf surface of oat seedlings (cv. ‘Brooks’) treated with treated with Aphanocladium album (Aa100) crude extract. Notice black telia beginning to form near the midvein of the leaf.
FIG. 5. Formation of telia on oat seedlings (cv. ‘Brooks’) treated with treated with Aphanocladium album (Aa100) crude extract
FIG. 6. Formation of telia on oat seedlings (cv. ‘Brooks’) treated with treated with Aphanocladium album (Aa100) semi-purified fraction (Aa100-II-3)
FIG. 7. Formation of telia on oat seedlings (cv. ‘Brooks’) treated with treated with pure compound (Ooosporein) isolated from Aphanocladium album (Aa100)
FIG. 8. Teliospores and urediospore of Puccinia coronata

### Synthesis of Oosporein

Based on initial results, the active compound is isolated from the fermented broth of A. album. However, from the economic point of view, isolation of large amounts of the compound from the fermentation broth of A. album is both time consuming, expensive and would require specialized facilities to scale up the 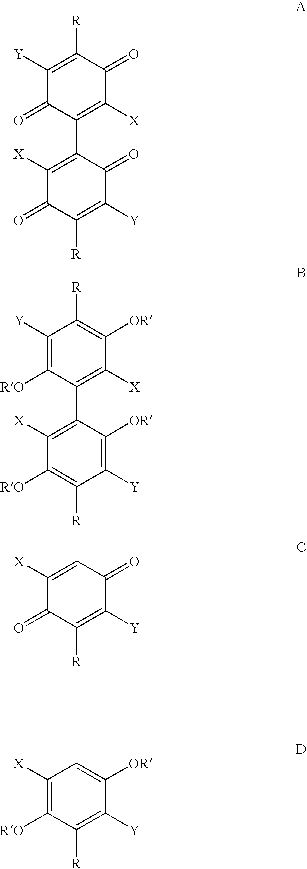
fermentation and down stream processing. In addition, the fermentation broth contains a small amount (0.84%) of the compound. Therefore it is desirable to look for derivatives of the compound to identify more potent activity to facilitate total synthesis of the active compound. Since the metabolite that induces conversion of urediniospores to teliospores has been identified as oosporein, a rather simple, low molecular weight compound, this offers the opportunity of preparing this compound synthetically. Additionally, this allows preparation and evaluation of a number of oosporein analogs to determine if any such compounds show improved teliaspore inducing activity. Structural features which will be varied are illustrated as structures A-D and will be briefly outlined below. Specific synthetic approaches are described in subsequent sections.
Variations in Alkyl Chain Length
Though oosporein (Structure A, R=Me; X═YαOH) possesses simple methyl groups at either end of the central bisquinone, other natural products have been identified which possess longer chains. For example, biembelin (1), has been isolated from the fruits of Rapanea melanphloes (Midiwo and Ghebrenieskel, 1993) while diboviquinone (2) has been isolated from the fungus Suillus bovines (Kasuga et al. 1995; Edwards and Beaumont, 1971). A series of oosporein analogs are prepared in which the length of the alkyl chain are varied. Compounds with chain lengths ranging from ethyl to octadecyl are prepared. Additionally, some simple branched chain analogs (R=i-Bu, isopentyl, isohexyl) are investigated, as is the incorporation of an aromatic 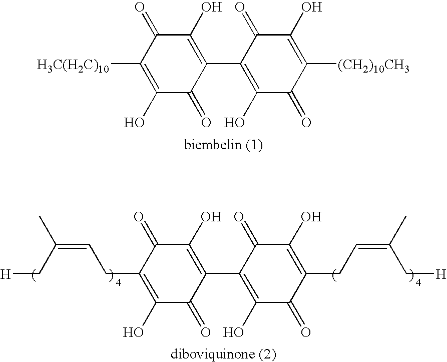
ring into this side chain (R=benzyl). Compounds possessing a variety of substituted benzene rings are prepared as a means of evaluation of the effect both steric and electronic parameters would have on the bioactivity.
Variations in Oxidation State

Reduced analogs of oosporein, including both bisquinones (Structure A, X═Y═H) and bishydroquinones (Structure B, R′═H; X═Y═OH) are prepared. Variations in the chain length of R are investigated in this series of compounds as well.

Variations in Etherification

Etherification of the hydroxy groups present in oosporein and its analogs are investigated. (Such compounds are actually more likely to be prepared as precursors to the hydroxy-substituted compounds-see specific syntheses). Initially, methyl ethers are synthesized of compounds in various oxidation states (Structure A, X═Y═OMe; Structure B, R′=Me, X═Y═OMe, and also, R′=Me, X═Y═H), but if such ethers show superior activity relative to their hydroxylated congeners, variations in these alkoxy groups would also be investigated. Both fully methylated and partially methylated structures (e.g., Structure B, R′=Me, X═OH, Y═OMe, etc.), would be synthesized. Once again, compounds with R groups of varying length are prepared and evaluated for their teliospore inducing activity.

“Monomeric” Analogs of “Dimers”

There exist a number of alkylated quinone natural products of general structure “C” (see above) that can be thought of as “half” of the oosporein structure. One of the most well-known is embelin (3), which has been shown to possess significant biological activity (Nikolovska et al. 2004; Chitra et al. 1994). “Monomeric” analogs of the “dimeric” compounds discussed above are prepared and evaluated. Comparison of the biological activity of these two sets of compounds (“monomer” vs. “dimer”) allows us to determine the necessity of the dimeric structure for induction of the conversion of urediniospores to teliospores. As various reduced and/or alkylated forms of these quinones (e.g., Structure C, X═Y═OMe and variations on Structure D), are also known (Poigny et al. 1998; Arot et al. 2003; Pfeifer and Gerlach, 1995) and have been found to possess biological activity (Meazza et al. 2002; Haraguchi et al. 1996) a series of such compounds are prepared and evaluated as part of the synthetic studies. 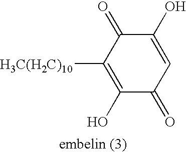
Syntheses of Oosporein: There is a limited amount of literature on syntheses of oosporein. One of the earliest was reported by Birch and Fryer (Birch and Fryer, 1969) in which the corresponding hydroquinone, isolated as a metabolite from a fungus, was oxidized with chromic acid to give oosporein in unspecified yield. A few years later, Dallacker and Löhnert (1972) prepared oosporein starting from 4,5-dimethoxy-1,2-methylenedioxybenzene (4) Lithiation of (4) followed by treatment with methyl iodide produced (5) in 84% yield. Subsequent lithiation of this product, followed by oxidation with copper (II) chloride produced the dimeric species (6), albeit in only 40% yield. Oxidation with dilute nitric acid gave the ortho quinone dimer (7) in 93% yield, which was then hydrolyzed to oosporein in 90% yield.

Two different routes to the required 4,5-dimethoxy-1,2-methylenedioxybenzene (4) have been reported (FIG. 4). Piperonal (8) was oxidized with formic acid/hydrogen peroxide to give 3,4-methylenedioxyphenol (9) (sesamol) in 51% yield by means of a Bayer-Villiger oxidation (Dallacker et al. 1968). Oxidation of this phenol with Fremy's salt produced o-quinone (10) in 69% yield. Reduction of this quinone to hydroquinone (11) was accomplished in 81% yield by treatment with gaseous sulfur dioxide. Alkylation with dimethyl sulfate and potassium carbonate produced (4) in 73% yield.
Alternatively, oxidation of catechol (12) with lead oxide has been reported to produce quinone (13) in 51-60% yield (Dallacker and Loehnert, 1972). Reduction to the hydroquinone (14) with sodium dithionite was accomplished in 72% yield, and alkylation to give (4) using 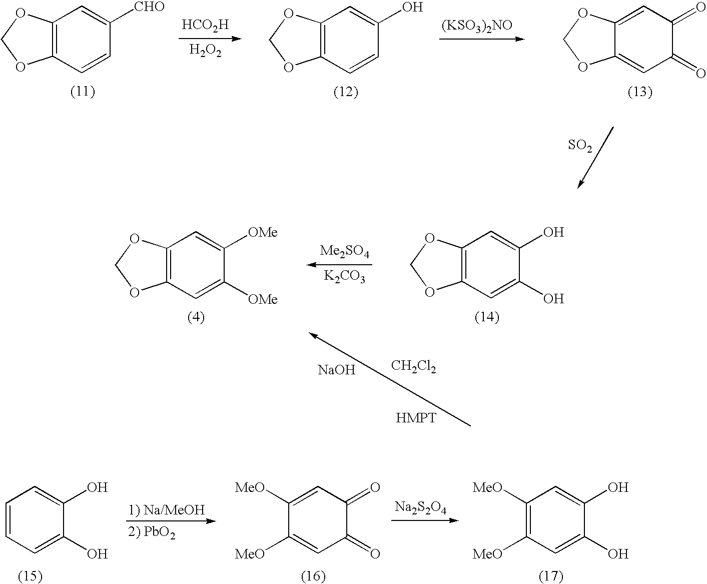
dichloromethane and HMPT proceeded in 79% yield.
A few years after publication of the paper by Dallacker and Löhnert, another synthesis of oosporein was reported by Posternak and coworkers (Kalamar et al. 1974). Starting from 1,2,4,5-tetramethoxybenzene (15), lithiation followed by alkylation with methyl iodide was used once again, giving toluene derivative (16) in 75% yield. Lithiation followed by treatment with molecular iodine afforded aryl iodide (17) in 51% yield. Iodide (17) could also be obtained in one pot from (15) by sequential treatment with four equivalents of butyllithium, one equivalent of iodine, then four equivalents of methyl iodide. The overall yield for this one pot reaction was 39%, which is roughly the same as was obtained by the two step method.

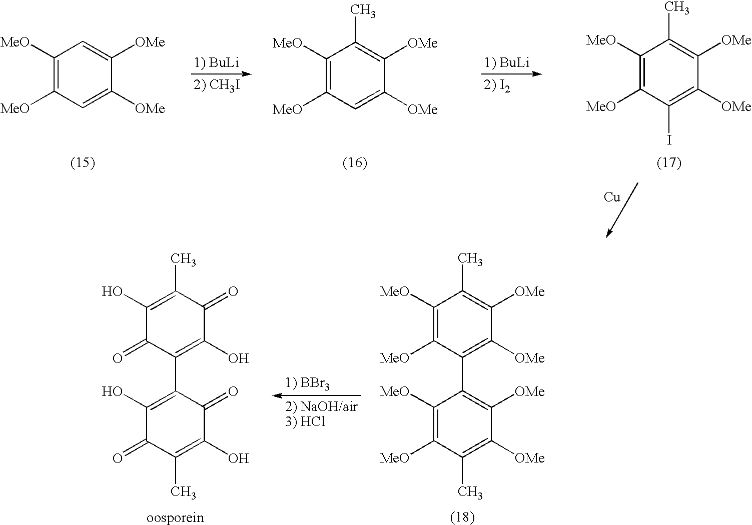

Biaryl (18) was prepared by heating (17) with copper at 250° C. for four hours, though the isolated yield was only 19%. Removal of the methoxy groups was accomplished using BBr₃, and air oxidation under basic conditions followed by acid work-up gave oosporein in unspecified yield.

As was the case with Dallacker and Löhnert's synthesis, preparation of the required starting material required several steps. In this case, 1,2,4,5-tetramethoxybenzene (15) was prepared by first treating purified p-benzoquinone (19) with freshly fused and powdered zinc chloride in absolute methanol to give 2,5-dimethoxybenzoquinone (20) in 64% yield (Benington et al. 1955). The crude product was reduced to the corresponding hydroquinone with sodium 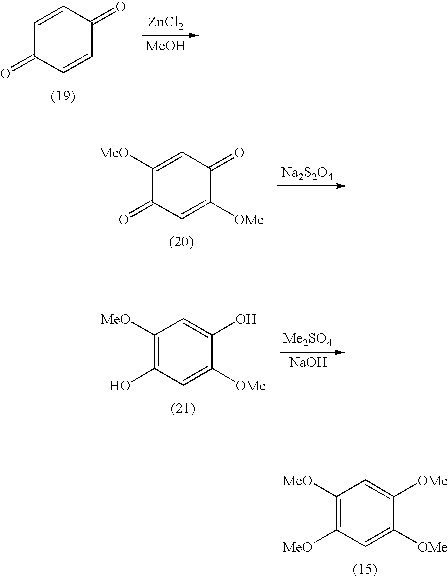
dithionite, giving a 78% yield of (21), which once again was used in the next step without purification. Alkylation to give (15) was accomplished in 86% yield using dimethyl sulfate.

A SciFinder Scholar® search failed to locate any newer syntheses of oosporein than the one described by Posternak (Kalamar et al. 1974). Oosporein is prepared by a method that combines some of the successes of these early syntheses with newer methods that have emerged in the intervening 30 years.

Routes to Syntheses of Oosporein

Like the earlier syntheses, some of our proposed routes to oosporein also utilize highly oxygenated benzene derivatives as key intermediates. A number of syntheses of such compounds have been reported since those cited by Posternak (Kalamar et al. 1974) and by Dallacker and Löhnert. For example, oxidation of hydroquinone with basic hydrogen peroxide produces 2,5-dihydroxy-1,4-benzoquinone (22) in 79% yield (Weider et al. 1985) This intermediate can be reduced to 1,2,4,5-tetrahydroxybenzene (23) either by tin and HCl (Weider et al. 1985) or by catalytic hydrogenation (Keegstra et al. 1996; Reddy et al. 2002) the former taking place in 75% yield, and the latter in quantitative (crude) yield (Weider et al. 1985). Alkylation of this tetrahydroxybenzene (23) to the corresponding tetramethoxybenzene (15) has also been reported to take place in 86% yield using potassium carbonate and methyl bromide (Keegstra et al. 1996). Similarly, acetalization of (23) to give (24) has been accomplished in 82% yield (Reddy et al. 2002).

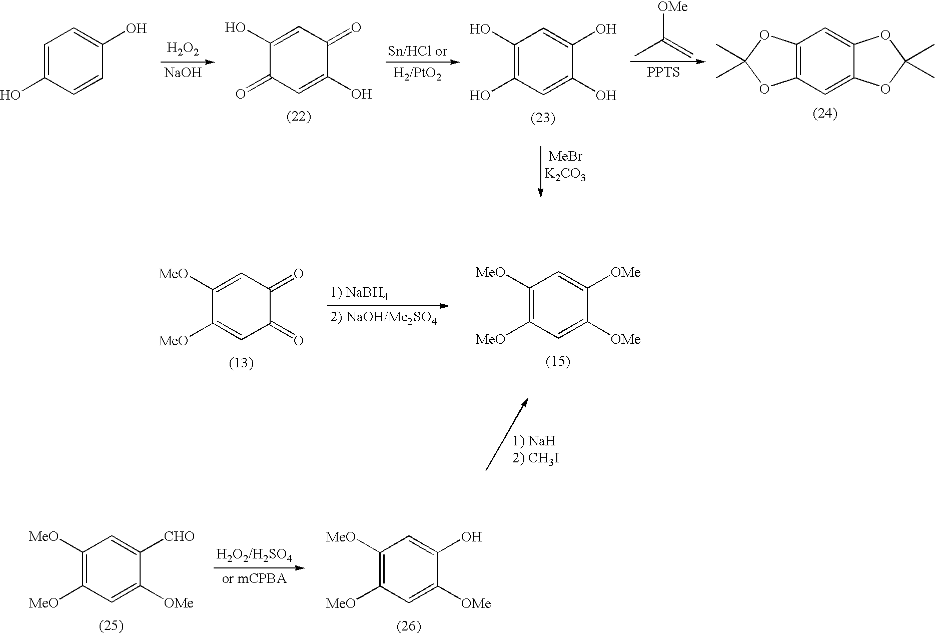

Other synthetic routes to 1,2,4,5-tetramethoxybenzene (15) have also been reported. For example, reduction of quinone (13) (obtained from treatment of catechol with lead oxide) with NaBH₄ followed by alkylation with dimethyl sulfate produces (15) in 75% yield for the two steps (Stahl and Waldmann, 1999) (A similar reaction using LiAlH₄ has also been reported, but the yields were slightly lower; Cole et al. 1980). Commercially available 2,4,5, trimethoxybenzaldehyde (25) has also been used as a starting material for the preparation of (15). Treatment of (25) with hydrogen peroxide in the presence of sulfuric acid produces the corresponding phenol (26) in 89% yield by means of a Bayer-Villiger reaction (Matsumoto et al. 1984). Use of mCPBA as the oxidant gives the same product in slightly higher (93%) yield (Poigny et al 1998). Alkylation with methyl iodide then produces (15) in 94% yield (Poigny et al. 1998).

Thus, a number of viable routes to key intermediate (15) are available. Due to the simplicity of the reactions and their high yields, our initial approach to the synthesis of (15) would be via aldehyde (25). For large scale preparation of this inter-mediate, the route starting from hydroquinone might become more attractive, owing to the lesser expense of hydroquinone relative to 2,4,5, trimethoxybenzaldehyde (Aldrich Catalog, 2004-05). Routes starting from catechol would be considered with alternatives to lead based on oxidants being identified.

With 1,2,4,5-tetramethoxybenzene (15) in hand, the next step in our synthesis is lithiation followed by alkylation to give methylated adduct (16). Not only is this exact transformation precedented (Kalamar et al. 1974) but similar alkylations have been reported 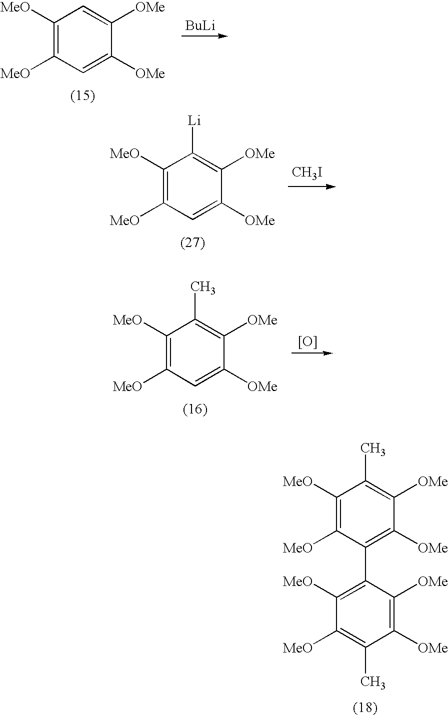
using a wide range of alkylating agents (Poigny et al. 1998a; 1998b; 1999; Bruggemann et al. 2001).

Oxidative coupling of (16) to yield (18) is investigated. A number of methods for the coupling of electron-rich aromatics to give biaryls have been reported recently. For example, phenyliodine(III) bis(trifluoroacetate) oxidizes 1,2 4 trimethoxybenzene (28) to the corresponding biphenyl derivative (29) in 92% yield (Tohma et al. 2001). Similarly, commercially available (Aldrich Catalog, 2004-05) 2,5-dimethoxyltoluene (30) has been oxidized to biaryl (31), also in 92% yield using the same reagent. Other oxidations on similar substrates have also been reported (Takada et al. 1998; Hamamoto et al. 2002).

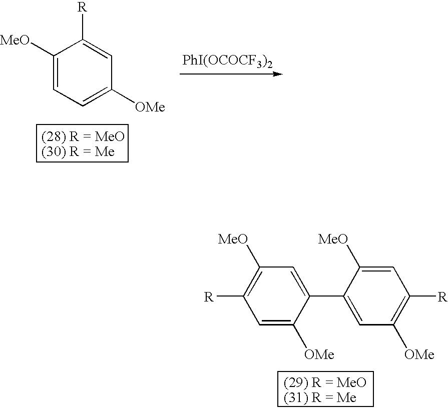

A more readily available oxidant that has been used to couple electron rich aromatics is molybdenum pentachloride (Kramer et al. 2003). For example, treatment of 3,4-dimethoxytoluene with MoCl₅ provided a 69% yield of biaryl (32) in only 40 minutes at room temperature. Even more amenable to scale-up is use of iron (III) chloride as an oxidant, which has been shown to effect this same transformation in 64% yield when adsorbed on silica gel 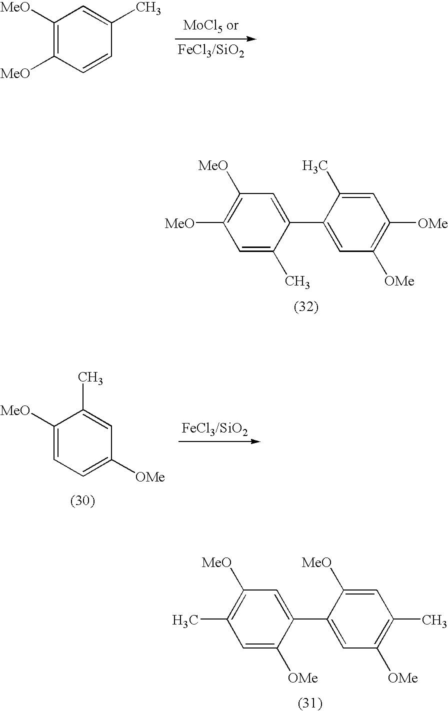
(Jempty et al. 1981). Similarly, (31) could be prepared in 82% yield using the same reagent, starting from 2,5-dimethoxyltoluene (30).

Adsorbtion of FeCl₃ on solid supports often improves the yields and/or purities of products obtained from oxidative phenolic couplings. For example, the 1,1′-bi-2-naphthol (BINOL) obtained from the oxidation of 2-naphthol by FeCl₃ on alumina is of much higher purity than that obtained from the analogous solution phase oxidation of 2-naphthol using FeCl₃ (Love and Bills, 2002). This same methodology has been applied to the coupling of naphthyl ethers (Love and Bills, Unpublished).

Other coupling methods are investigated to provide the desired intermediate (18). For example, sequential treatment of anisole with butyllithium (to achieve ortholithiation) followed by zinc iodide, then N-chlorosuccinimide and catalytic Pd(Ph₃)₄ provided 2,2′-dimethoxybiphenyl (33) in 84% yield (Hossain et al. 2001). This same product has been obtained in identical yield by reaction of 2-iodoanisole with zinc and triethylammonium formate (Abiraj et al. 2004). Either of these methods can be used to prepare (18) from the corresponding intermediates (27) or (17).

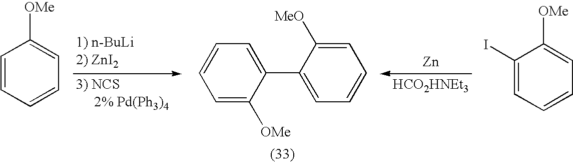

With biaryl (18) in hand, oxidative conversion to oosporein should be relatively straightforward. Indeed, this precise transformation was carried out by Posternak and coworkers (Kalamar et al. 1974). Other, more recent oxidative methods involve, for example, a number of alkylated tetramethoxybenzene derivatives have been treated with ceric ammonium nitrate to give the corresponding dimethoxyquinones (34) (Bruggemann et al. 2001; Poigny et al. 1999). This same methodology will convert (18) into (35), which can then be hydrolyzed to oosporein.

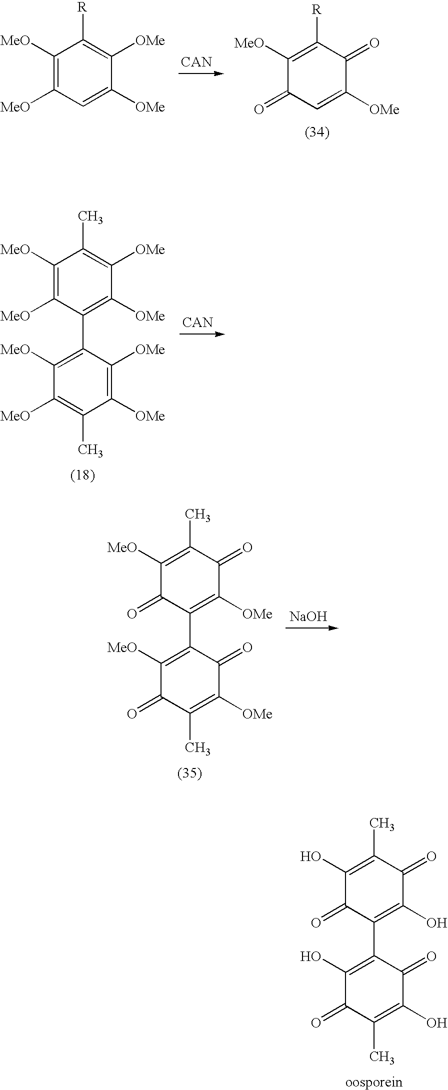

Simple hydrolysis followed by air oxidation has been shown to convert bisketal (36) into dihydroxyquinone (37) in quantitative yield (Weider et al. 1985). This same methodology is used for the preparation of oosporein if either bisketal (24) (Reddy et al. 2002) or (38) (Weider et 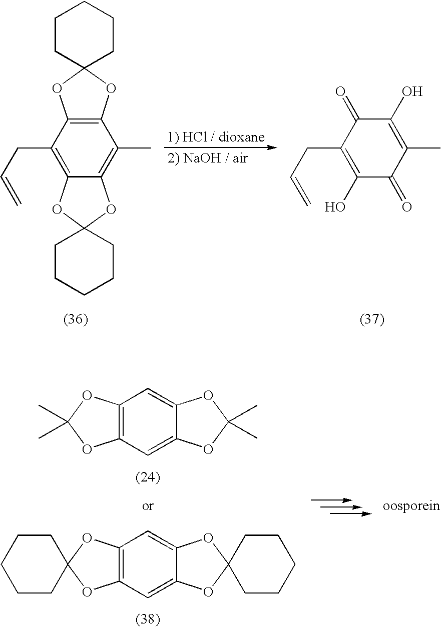
al. 1985) were to be used in place of 1,2,4,5-tetramethoxybenzene in the previously described synthetic sequence.
Other Routes of Syntheses of Oosporein

Other synthetic routes less closely related to the literature syntheses may provide quicker and/or higher yielding approaches to oosporein. In many cases oxidative phenolic couplings can be facilitated by tethering the two aryl components, thus making the coupling reaction intramolecular. For example, treatment of siloxane (39) with phenyliodine(III) 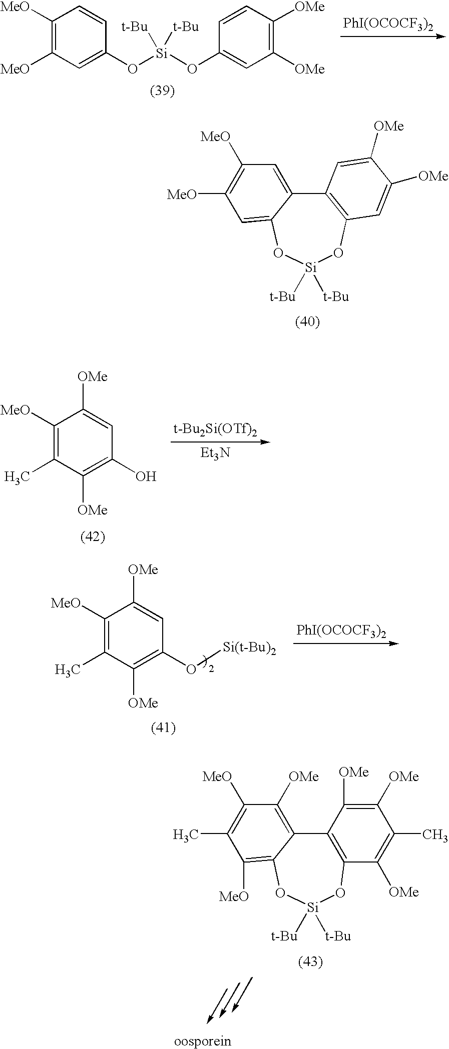
bis(trifluoroacetate) produces coupled product (40) in 94% yield (Hamamoto et al. 2002). Preparation of siloxane (41) from known (Maruyma et al. 1986) phenol (42) followed by oxidative coupling produces (43), which can be converted to oosporein by methods analogous to those already described.

Alternatively, it has been demonstrated that oxidation of 2,5-dimethoxytoluene (30) with ceric ammonium nitrate leads directly to bisquinone (44) in 85% yield (Jacob et al. 1976). Since treatment of quinones with bromine is known to give dibromoquinones, (Frank et al. 1950) one would expect that bromination of bisquinone (44) would lead to (45), which could then be hydrolyzed to yield oosporein. Since 2,5-dimethoxytoluene is commercially available (Aldrich Catalog, 2004-05) this would represent an especially short and efficient synthesis of oosporein.

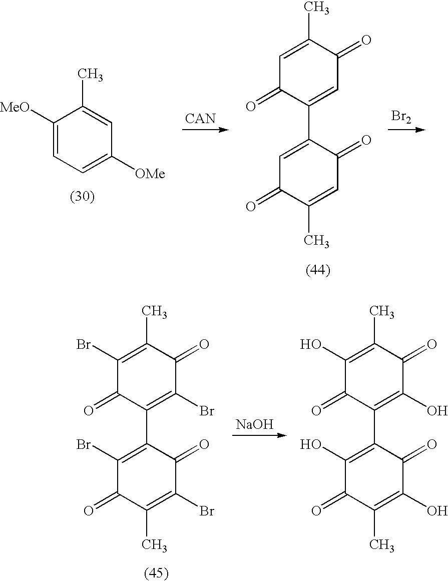

Similarly, both 2,5-dihydroxytoluene (46) and 2,3,5-trihydroxytoluene (47) have been oxidized to the corresponding bisquinones (44) and (48) in 75% and 65% yield, respectively using oxygen and a copper-propanediamine complex (Paraskevas et al. 1988). Bromination and hydrolysis of either of these products should once again lead to oosporein. 2,5-Dihydroxytoluene (46) is commercially available, (Aldrich Catalog, 2004-2005) while 2,3,5-trihydroxytoluene (47) is not. 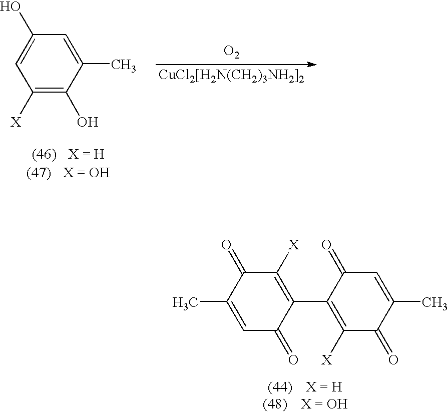
Preparation of Derivatives of Oosporein

The synthetic approaches described below offer the advantage of allowing investigation of the introduction of other alkyl groups onto the oosporein framework, making such compounds available for biological evaluation. Thus, oosporein derivatives (49) possessing R groups of increasing chain length (ethyl, propyl, etc.) can be prepared and evaluated to determine the effects of increasing lipophilicity and/or steric bulk on the biological profile. Synthesis of such derivatives simply require preparation of the requisite alkylated electron-rich benzene derivatives as starting materials. Such alkylations either proceed via direct 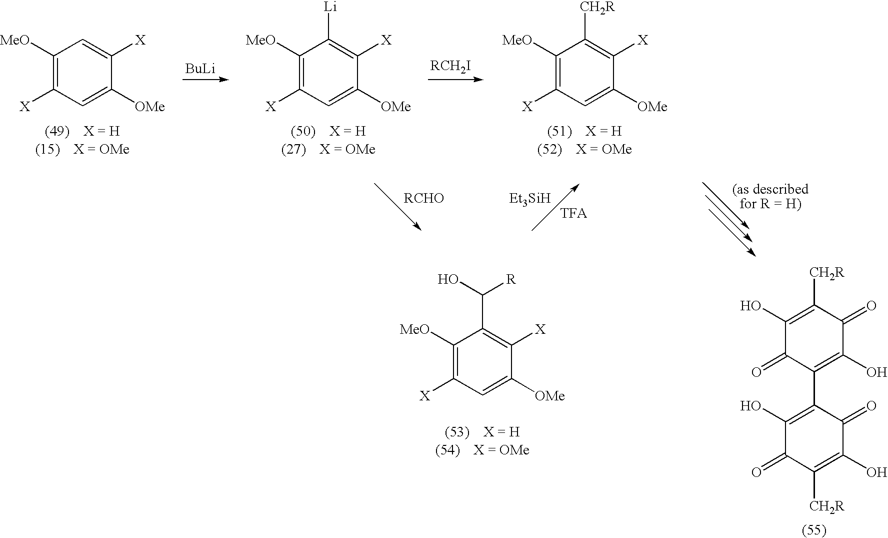
alkylation of aryllithiums or by reaction of aryllithiums with aldehydes, followed by reduction. Both of these processes are well-precedented (Poigny et al 1998a; Weider et al. 1985; Poigny et al. 1999; Bernet et al. 2003; Kawai et al. 2000; Arndt et al. 2001).

As seen previously, reduced forms of oosporein would be prepared as part of some potential routes to this natural product. These would also be evaluated for biological activity, as would the fully and partially methylated ether derivatives of such compounds. Since it is unknown which structural features of oosporein are required for its activity, results of initial testing would guide further derivative synthesis and evaluation.

Characterization Methods

All compounds prepared as part of this work are fully characterized (IR, ¹H and ¹³C NMR; mass spectrometry when necessary). Though no longer a normal characterization method for most organic compounds, use of UV/V is spectroscopy proves useful in the identification of many of the products and intermediates in this synthetic effort, as many of these compounds possess significant chromophores. Initial efforts focus on the synthesis of known compounds, (as these are easier to positively identify) and serve as a validation of the synthetic methods used. All previously unknown compounds prepared are also characterized by elemental analysis and/or high resolution mass spectrometry.

In a more recent development of the invention, work toward the synthesis of oosporein has progressed via several approaches. One set of approaches to the synthesis of oosporein utilizes 2,5-dimethoxytoluene (1) (commercially available, and available in one step in high yield from the very inexpensive starting material methylhydroquinone) as the initial starting material. Proper choice of oxidant and reaction conditions allowed preparation of either biphenyl derivative (2) or diquinone (3), both in high yield and good purity (Scheme 1).

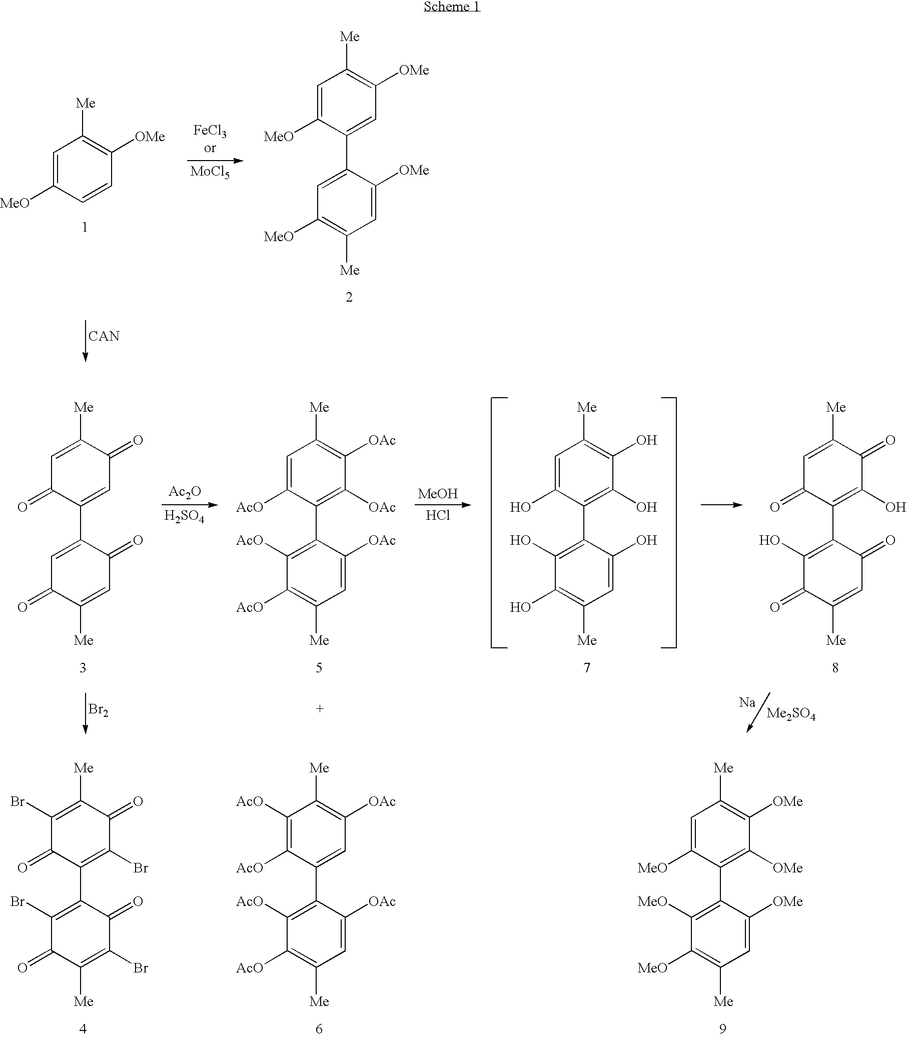

Initial efforts to introduce oxygen or readily oxidizable groups into (2) had little success, however, the approach by looking at acylation followed by a Baeyer-Villiger reaction as a way of introducing additional oxygenated functionality into (2).

Bromination of (3) to yield tetrabromo compound (4) proceeded in high yield using bromine in acetic acid. Although the simple displacement of the bromides with hydroxide should theoretically lead to oosporein (such displacements are well precedented in other quinone derivatives), this in fact is not a viable approach, as it has been shown that oosporein decomposes upon prolonged treatment with base. Thus, conditions sufficiently vigorous to effect bromine displacement also lead to the decomposition of the initially formed oosporein. We have tried to circumvent this problem by using a variety of other nucleophiles, but so far, without success. Very recently encouraging results have been obtained using azide as the nucleophile, but as of this writing this product has not yet been fully characterized. If indeed it is the expected tetraazido compound, oosporein should be accessible via reduction of the azide groups to primary amines.

Yet another approach to oosporein has involved a Thiele-Winter reaction of diquinone (3) to give hexaacetate (5). Although the product mixture contains a small amount of the isomeric compound (6), this unsymmetrical diquinone can be easily removed by simple trituration with methanol, leaving pure (5) in good yield. (The structure of (5) has been confirmed by X-ray diffraction of a single crystal). Hexaacetate (5) can be readily hydrolyzed with acidic methanol to give (7), but attempts to introduce either oxygen- or nitrogen-based functionality directly into either (5) or (7) have thus far been unsuccessful. Simple air oxidation, does, however, provide a nearly quantitative yield of the natural product phoenicin (8). Although it might appear that a second Thiele-Winter reaction conducted on (8) should lead to the corresponding octaacetate, literature precedent has shown this not to be the case. Instead, intramolecular ring closure to give dibenzofuran derivatives dominates. This has in fact been a recurring theme throughout much of this research-reactions which are well-precedented in simple quinones fail to give the intended product when applied to diquinones due to intramolecular ring closing reactions.

Nevertheless, numerous attempts have been made to introduce either oxygen- or nitrogen-based functionality onto the unsubstituted carbons of (8), but have failed to give appreciable yields of the desired products in sufficient purity. Likewise, attempts to modify the hydroxyl groups in (8) to lower the likelihood of their participation in intramolecular cyclizations have not yet met with success. We have, however, recently succeeded in producing hexamethoxy derivative (9) by treatment of (8) with sodium and dimethyl sulfate in isopropyl alcohol (attempts to alkylate (7) directly were unsuccessful). Oxidation of (9) under a variety of conditions will be explored as a means of producing oosporein.

The early successes in producing compounds (3), (4), (5) and (8) encouraged investigation of synthetic routes which utilized these intermediates. Not only could these compounds be produced in high yield, but they were also obtained in high states of purity without need of chromatography or recrystallization. This coupled with the fact that no air-sensitive reagents (such as organometallics) were necessary for their synthesis made these routes very amenable to potential scale-up, a factor which was exceptionally attractive owing to the eventual desire to do field testing of such compounds. Nevertheless, preparation of usable quantities of oosporein by these methods has proven much more difficult than imagined, and thus alternative approaches to oosporein (and related compounds) are also being investigated. Although these alternative approaches do not offer the advantage of ease of scale-up, they might be useful in producing small quantities of oosporein and its analogs for laboratory testing which could be conducted while problems with the former approaches are being resolved.

Since many of the difficulties encountered with the previous approaches involved complications due to intramolecular ring closure, one idea was to prepare a suitably functionalized monocyclic precursor and then dimerize it to give key intermediates in oosporein synthesis. Toward this end, commercially available 1,2,4-trimethoxybenzene (10) has been lithiated with n-butyllithium followed by reaction with methyl iodide to give trimethoxytoluene (11) in near quantitative yield. Efforts are now being directed toward oxidation of (11) to give quinones (12) and (13), either of which could be useful in the synthesis of oosporein.

We have also produced (15) and (16) by reaction of commercially available 2,6-dimethoxytoluene (14) with either one or two equivalents of bromine. Attempts to convert (15) into quinone (17) have thus far been unsuccessful, as have attempts to convert (16) into (18) by copper-catalyzed methoxylation.

Finally, we have followed a series of literature procedures for the preparation of (18) starting from commercially available aldehyde (19). This has allowed us to prepare small quantities of (18), which is a known intermediate in a literature synthesis of oosporein. These reactions are currently being scaled up in order to produce larger quantities of (18) so that it may then be carried on to oosporein following either literature procedures or variations on them.

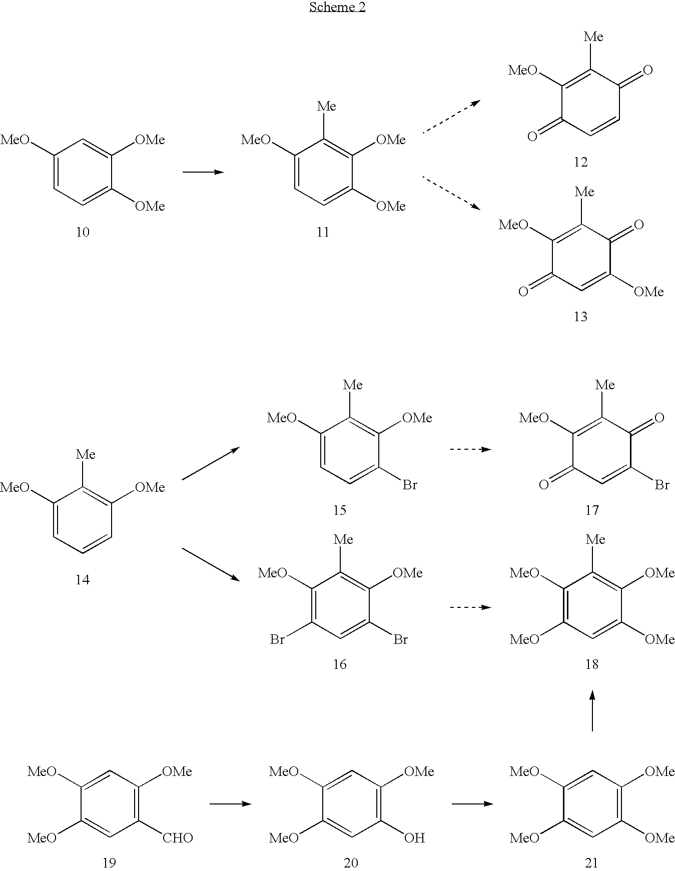

For further background, the following are some of the references useful to the understanding of the invention.
Boufatah, N.; Gellis, A.; Maldonado, J.; Vanelle, P. “Efficient microwave-assisted synthesis of new sulfonylbenzimidazole-4,7-diones: heterocyclic quinines with potential antitumor activity.” Tetrahedron (2004), 60, 9131-9137.
Cornelis, A.; Laszlo, P. “Clay-supported copper(II) and iron(III) nitrates: novel multi-purpose reagents for organic synthesis.” Synthesis (1985), 100, 909-918.
Dwyer, C. L.; Holzapfel, C. W. “The nitration of electron-rich aromatics.” Tetrahedron (1998), 54, 7843-7848.
Frank, R. L.; Clark, G. R.; Coker, J. N. “Synthesis of vulpinic acid from polyporic acid.” Journal of the American Chemical Society (1950), 72, 1824-6.
Gigante, B.; Prazeres, A. O.; Marcelo-Curto, M. J. “Mild and selective nitration by “claycop”.” Journal of Organic Chemistry (1995), 60, 3445-3447.
Ito, T., Ikemoto, t.; Yamano, T.; Mizuno, Y.; Tomimatsu, K. “Practical synthesis of (R)-(+)-6-(1,4-dimethoxy-3-methyl-2-naphthyl)-6-(4-hydroxyphenyl)hexanoic acid: a key intermediate for a therapeutic drug for neurodegenerative diseases.” Tetrahedron: Asymmetry (2003), 14, 3525-3531.
Jacob, P., III; Callery, P. S.; Shulgin, A. T.; Castagnoli, N., Jr. “A convenient synthesis of quinines from hydroquinone dimethyl ethers. Oxidative demethylation with ceric ammonium nitrate.” Journal of Organic Chemistry (1976) 41(22), 3627-3629.
Jempty, T. C.; Gogins, K. A. Z.; Mazur, Y.; Miller, L. L. “Iron trichloride/silicon dioxide reacts as oxidant or Lewis acid with phenol ethers.” Journal of Organic Chemistry (1981), 46(22), 4545-4551.
Kalamar, J.; Steiner, E.; Charollais, E.; Posternak, T. “Biochemistry of the lower fungi. VIII. Chemical synthesis of diquinonic pigments.” Helvetica Chimica Acta (1974), 57(8), 2368-2376.
Kramer, B.; Frohlich, R.; Bergander, K.; Waldvogel, S. R. “The O-substitution pattern of the MoCl5-mediated oxidative aryl-aryl coupling reaction.” Synthesis (2003), (1) 91-96.
Lemarchand, A.; Bach, T. “Synthesis of a para-quinone macrolactam related to geldanamycin by ring closing metathesis.” Tetrahedron (2004), 60, 9659-9673.
Love, B. E.; Bills, R. A., “Facile synthesis of binol in the absence of solvent.” Synthetic Communications (2002), 32 (13), 2067-2073
Paraskevas, S. M.; Konstantinidis, D.; Vassilara, G. “Oxidation of hydroquinones with oxygen in the presence of bis(1,3-propanediaminato)copper(II) chloride.” Synthesis (1988), (11), 897-899.
Poigny, S.; Guyot, M.; Samadi, M. “Total synthesis of maesanin and analogs.” Tetrahedron (1998) 54(49), 14791-14802.
Shi, M and Cui, S. “A new method for mitration of phenolic compounds.” Adv. Synth. Catal. (2003), 345, 1197-1202.
Sudhir, U.; James, B.; Joly, S.; Nair, M. S. “Diels-alder reactivity of 2-(bromomethyl)-1,4-quinone and 2-bromo-5-(bromomethyl)-1,4-quinone with cyclopentadiene and the synthesis of new substituted pentacyclic systems.” Res. Chem. Intermed. (2003), 29(5), 523-532.
   In summary, a number of viable and well precedented reaction pathways exist for the preparation of oosporein and structurally related compounds. These provide ready access to usable quantities of these compounds for investigation of their biological activity. Accordingly, the invention is now described in the appended claims in which it is set forth in a non-limiting manner.

What is claimed:

1. A method of controlling rust disease in plants, comprising,
recovering low molecular weight metabolites produced by Aphanocladium album mycoparasites from rust urediniospores; and
applying an effective amount of said metabolite to plants at risk of acquiring rust disease to convert infective urediniospores that cause rust disease into non-infective teliospores.

2. The method of claim 1, wherein said plants comprise at least one of oat, barley, soybean, wheat, peanut, coffee, ornamental plants and pine.

3. The method of claim 1, wherein said metabolite comprises oosporein and related compounds.

4. The method of claim 1, wherein the metabolite is reacted with another substance to result in diquinone prior to application to the plants.

5. The method of claim 4, wherein said diquinone is subjected to a Thiele-Winter reaction to result in hexaacetate prior to application to the plants.

6. A method of controlling rust disease in plants, comprising,
recovering low molecular weight metabolites produced by Beauveria brongniartii mycoparasites from rust urediniaospores; and
applying an effective amount of said metabolite to plants at risk of acquiring rust disease to convert infective urediniospores that cause rust disease into non-infective teliospores.

7. The method of claim 6, wherein said plants comprise at least one of oat, barley, soybean, wheat, peanut, coffee, ornamental plants and pine.

8. The method of claim 6, wherein said metabolite comprises oosporein and related compounds.

9. The method of claim 6, wherein the metabolite is reacted with another substance to result in diquinone prior to application to the plants.

10. The method of claim 6, wherein said diquinone is subjected to a Thiele-Winter reaction to result in hexaacetate prior to application to the plants.

11. A composition for controlling rust disease in plants, comprising low molecular weight metabolites produced by one of Aphanocladium album and Beauveria brongniartii parasites which have been recovered therefrom.

12. The composition of claim 11, wherein said composition is effective against rust disease in at least one of oat, barley, soybean, wheat, peanut, coffee, ornamental plants and pine.

13. The composition of claim 1, wherein said metabolite comprises oosporein and other compounds produced by Aphanocladium album.

14. The composition of claim 1, wherein the metabolite has been reacted with another substance to result in diquinone for preparation to be applied to plants.

15. The composition of claim 14, wherein said diquinone has been subjected to a Thiele-Winter reaction to result in hexaacetate for preparation to be applied to the plants.
